(19) United States
(12) Patent Application Publication
(10) Pub. No.: US 2025/0064930 A1
(43) Pub. Date: Feb. 27, 2025

(54) ANTI-IL-31RA ANTIBODY-CONTAINING FORMULATIONS

(71) Applicant: Chugai Seiyaku Kabushiki Kaisha, Tokyo (JP)

(72) Inventors: Daisuke Kameoka, Tokyo (JP); Toru Yoshizawa, Tokyo (JP); Megumi Numata, Tokyo (JP); Hitoshi Sasaki, Tokyo (JP); So Yamaguchi, Tokyo (JP); Hiroko Murata, Shizuoka (JP); Naoka Hironiwa, Synapse (SG)

(73) Assignee: Chugai Seiyaku Kabushiki Kaisha, Tokyo (JP)

(21) Appl. No.: 18/948,772

(22) Filed: Nov. 15, 2024

Related U.S. Application Data
(63) Continuation of application No. 17/686,536, filed on Mar. 4, 2022, now Pat. No. 12,171,830.
Application No. 17/686,536 is a division of application No. 17/573,991, filed on Jan. 12, 2022, now Pat. No. 11,723,976.
Application No. 17/573,991 is a division of application No. 17/346,421, filed on Jun. 14, 2021, now Pat. No. 11,260,125.
Application No. 17/346,421 is a continuation of application No. 17/058,399, filed on Nov. 24, 2020, now abandoned.

(30) Foreign Application Priority Data
Nov. 20, 2019 (JP) 2019-209851

(51) Int. Cl. A61K 39/395; A61K 9/08; A61K 9/19; A61K 39/00; A61K 47/10; A61K 47/18; A61K 47/26; C07K 14/715; C07K 16/28

(52) U.S. Cl. CPC A61K 39/39591; A61K 9/08; A61K 9/19; A61K 39/3955; A61K 47/10; A61K 47/18; A61K 47/183; A61K 47/26; C07K 16/2866; A61K 2039/505; A61K 2039/54; C07K 14/7155; C07K 2317/56; C07K 2317/565; C07K 2317/76

(57) ABSTRACT

In one non-limiting embodiment, the present disclosure relates to lyophilized formulations containing an IL-31 antagonist (for example, an anti-IL-31RA antibody) as an active ingredient, the lyophilized formulations further containing arginine and/or a salt thereof and sucrose and/or trehalose. In another non-limiting embodiment, the present disclosure relates to solution formulations containing an IL-31 antagonist as an active ingredient, the solution formulations further containing arginine and/or a salt thereof. In other non-limiting embodiments, the present disclosure relates to methods for stabilizing an antibody (for example, an anti-IL-31RA antibody) in an antibody-containing formulation, methods for suppressing antibody aggregation (aggregate formation) in an antibody-containing formulation, and methods for reducing components with charge heterogeneity in an antibody-containing formulation, the methods being characterized in that the formulation is prepared to contain arginine and/or a salt thereof, and/or sucrose and/or trehalose.

### CROSS-REFERENCE TO RELATED APPLICATIONS

This application is a continuation application of U.S. application Ser. No. 17/686,536, filed on Mar. 4, 2022, which is a divisional application of U.S. application Ser. No. 17/573,991, filed on Jan. 12, 2022 (now U.S. Pat. No. 11,723,976), which is a divisional application of U.S. application Ser. No. 17/346,421, filed on Jun. 14, 2021 (now U.S. Pat. No. 11,260,125), which is a continuation application of U.S. application Ser. No. 17/058,399, filed on Nov. 24, 2020 (now abandoned), which is the National Stage of International Application No. PCT/JP2020/043125, filed on Nov. 19, 2020, which claims the benefit of Japanese Application No. 2019-209851, filed on Nov. 20, 2019. The contents of the applications referenced above are incorporated herein by reference in their entireties.

### SEQUENCE LISTING

This application contains a Sequence Listing that has been submitted electronically as an XML file named C1-A1925P-USD4sq.xml. The XML file, created on Nov. 11, 2024, is 17,173 bytes in size. The material in the XML file is hereby incorporated by reference in its entirety.

### TECHNICAL FIELD

The present invention relates to stable formulations comprising an interleukin-31 (IL-31) antagonist (for example, a monoclonal antibody having the function to bind to IL-31 receptor A (IL-31RA) and to inhibit binding of IL-31 to IL-31RA).

### BACKGROUND ART

Monoclonal antibodies having the function to bind to interleukin-31 (IL-31) receptor A (IL-31RA) and to inhibit binding of IL-31 to IL-31RA were identified (PTLs 1-3). Anti-IL-31RA antibody Nemolizumab (CIM331) is a humanized IgG2 antibody which acts as an IL-31 antagonist to inhibit functions of IL-31 which is known as a pruritus-inducing cytokine; therefore, clinical trials on atopic dermatitis patients are currently ongoing.

Aiming to improve stability, numerous lyophilized formulations for therapeutic proteins such as antibodies have been developed so far (NPL 1). As additives for lyophilized formulations for non-antibody therapeutic proteins, sugars such as sucrose and trehalose are commonly used (NPL 2), and there are reports of cases where arginine was used as a stabilizing agent (PTLs 4 and 5).

However, for a formulation comprising an anti-IL31RA antibody (CIM331) which is an IL-31 antagonist, there is no report on a stable formulation where aggregate formation and/or components with charge heterogeneity which largely affects the antibody stability is suppressed.

### CITATION LIST

### Patent Literature

[PTL 1] WO2007/142325
[PTL 2] WO2009/072604
[PTL 3] WO2010/064697
[PTL 4] U.S. Pat. No. 6,887,852B1
[PTL 5] WO2010/148253

### Non-Patent Literature

[NPL 1] Pharm Res. 1997; 14 (8): 969-975.
[NPL 2] J Pharm Sci. 1998; 87 (11): 1412-1420.

### SUMMARY OF THE INVENTION

### Technical Problem

An objective of the present invention is to provide stable formulations that comprise an anti-IL-31RA antibody (CIM331) which is an IL-31 antagonist as an active ingredient.

### Solution to Problem

The present inventors have conducted dedicated research to achieve the above-described objective and, as a result, found that the addition of arginine or a salt thereof to formulations comprising the above-described IL-31 antagonist (an anti-IL-31RA antibody (CIM331) in particular) increases stability of the formulations comprising the IL-31 antagonist regardless of the presence or absence of a lyophilization step. It was also found that the addition of sucrose or trehalose to the formulations increases stability of the formulations comprising the IL-31 antagonist when the formulations are in a lyophilized state. It was further found that the addition of a nonionic surfactant to the formulations increases stability of the formulations comprising the IL-31 antagonist when the formulations are in a solution state.

In a non-limiting embodiment, the present disclosure relates to the following.
[1] A lyophilized formulation comprising an IL-31 antagonist as an active ingredient, wherein the formulation comprises arginine and/or a salt thereof, sucrose and/or trehalose.
[2] The lyophilized formulation of [1], wherein the arginine and/or salt thereof is arginine-hydrochloride, arginine-aspartate, or arginine-glutamate.
[3] The lyophilized formulation of [1] or [2], wherein the arginine and/or salt thereof is arginine-hydrochloride (Arg-HCl).
[4] The lyophilized formulation of any one of [1] to [3], further comprising Tris buffer as a buffer agent.
[5] The lyophilized formulation of [4], wherein the Tris buffer is tris(hydroxymethyl)aminomethane and/or a salt thereof.
[6] The lyophilized formulation of [5], wherein the tris(hydroxymethyl)aminomethane and/or salt thereof is tris(hydroxymethyl)aminomethane-hydrochloride (Tris-HCl), tris(hydroxymethyl)aminomethane-aspartate, tris(hydroxymethyl)aminomethane-glutamate, or tris(hydroxymethyl)aminomethane-acetate.
[7] The lyophilized formulation of [5] or [6], wherein the tris(hydroxymethyl)aminomethane and/or salt thereof is tris(hydroxymethyl)aminomethane-hydrochloride (Tris-HCl).
[8] The lyophilized formulation of any one of [1] to [7], further comprising Poloxamer 188 or polysorbate as a nonionic surfactant.
[9] The lyophilized formulation of [8], wherein the polysorbate is Polysorbate 20 or Polysorbate 80.
[10] The lyophilized formulation of any one of [1] to [9], wherein pH is 6 to 8 when the formulation is reconstituted in water.
[11] The lyophilized formulation of any one of [1] to [10], wherein pH is 6.5 to 7.5 when the formulation is reconstituted in water.
[12] The lyophilized formulation of any one of [1] to [11], wherein pH is 7 when the formulation is reconstituted in water.
[13] A solution formulation comprising an IL-31 antagonist as an active ingredient, wherein the solution formulation comprises arginine and/or a salt thereof.
[14] The solution formulation of [13], wherein the arginine and/or salt thereof is arginine-hydrochloride (Arg-HCl), arginine-aspartate, or arginine-glutamate.
[15] The solution formulation of [13] or [14], wherein the arginine and/or salt thereof is arginine-hydrochloride (Arg-HCl).
[16] The solution formulation of any one of [13] to [15], further comprising Tris buffer as a buffer agent.
[17] The solution formulation of [16], wherein the Tris buffer is tris(hydroxymethyl)aminomethane and/or a salt thereof.
[18] The solution formulation of [17], wherein the tris(hydroxymethyl)aminomethane and/or salt thereof is tris(hydroxymethyl)aminomethane-hydrochloride (Tris-HCl), tris(hydroxymethyl)aminomethane-aspartate, tris(hydroxymethyl)aminomethane-glutamate, or tris(hydroxymethyl)aminomethane-acetate.
[19] The solution formulation of [17] or [18], wherein the tris(hydroxymethyl)aminomethane and/or salt thereof is tris(hydroxymethyl)aminomethane-hydrochloride (Tris-HCl).
[20] The solution formulation of any one of [13] to [19], further comprising a nonionic surfactant, wherein the nonionic surfactant is Poloxamer 188 or polysorbate.
[21] The solution formulation of [20], wherein the polysorbate is Polysorbate 20 or Polysorbate 80.
[22] The solution formulation of any one of [13] to [21], wherein pH is 6 to 8.
[23] The solution formulation of any one of [13] to [22], wherein pH is 6.5 to 7.5.
[24] The solution formulation of any one of [13] to [23], wherein pH is 7.
[25] A lyophilized formulation comprising an IL-31 antagonist as an active ingredient, which is a composition resulting from lyophilizing a solution comprising:
   1 to 200 mg/mL IL-31 antagonist;
   1 to 200 mmol/L Tris buffer;
   4.5 to 1500 mmol/L arginine or a salt thereof;
   7.5 to 2500 mmol/L sucrose or trehalose; and
   0.01 to 5 mg/mL Poloxamer 188 or Polysorbate 20,
      wherein pH is 6 to 8 when the formulation is reconstituted in water:
[26] A lyophilized formulation comprising an IL-31 antagonist as an active ingredient, which is a composition resulting from lyophilizing a solution comprising:
   6 to 100 mg/mL IL-31 antagonist;
   6 to 20 mmol/L Tris buffer;
   45 to 150 mmol/L arginine or a salt thereof;
   75 to 250 mmol/L sucrose or trehalose; and
   0.15 to 0.50 mg/mL Poloxamer 188 or Polysorbate 20,
      wherein pH is 6 to 8 when the formulation is reconstituted in water:
[27] A lyophilized formulation comprising an IL-31 antagonist as an active ingredient, which comprises, per vial, cartridge, or syringe:
   1 to 800 mg IL-31 antagonist;
   0.1 to 40 mg tris(hydroxymethyl)aminomethane;
   0.8 to 400 mg arginine;
   3 to 1100 mg sucrose or trehalose; and
   0.01 to 7 mg Poloxamer 188 or Polysorbate 20,
      wherein pH is 6 to 8 when the formulation is reconstituted in water.
[28] A lyophilized formulation comprising an IL-31 antagonist as an active ingredient, which comprises, per vial, cartridge, or syringe:
   10 to 80 mg IL-31 antagonist;
   0.8 to 4 mg tris(hydroxymethyl)aminomethane;
   8 to 40 mg arginine;
   30 to 110 mg sucrose or trehalose; and
   0.1 to 0.7 mg Poloxamer 188 or Polysorbate 20,
      wherein pH is 6 to 8 when the formulation is reconstituted in water.
[29] The formulation of any one of [1] to [28], wherein the molar ratio of the arginine and/or salt thereof to the IL-31 antagonist is 220:1 to 1100:1, and/or the weight ratio of the arginine to the IL-31 antagonist is 0.3:1 to 1.3:1.
[30] The lyophilized formulation of any one of [1] to [12] and [25] to [28], wherein, between the sucrose or trehalose and the IL-31 antagonist, the molar ratio is 370:1 to 1840:1 and/or the weight ratio is 0.8:1 to 4.3:1.
[31] The lyophilized formulation of any one of [25] to [30], wherein the arginine and/or salt thereof is arginine-hydrochloride (Arg-HCl), arginine-aspartate, or arginine-glutamate.
[32] The lyophilized formulation of any one of [25] to [31], wherein the arginine and/or salt thereof is arginine-hydrochloride (Arg-HCl).
[33] The lyophilized formulation of [32], wherein the Tris buffer is tris(hydroxymethyl)aminomethane and/or a salt thereof.
[34] The lyophilized formulation of [33], wherein the tris(hydroxymethyl)aminomethane and/or a salt thereof is tris(hydroxymethyl)aminomethane-hydrochloride (Tris-HCl), tris(hydroxymethyl)aminomethane-aspartate, tris(hydroxymethyl)aminomethane-glutamate, or tris(hydroxymethyl)aminomethane-acetate.
[35] The lyophilized formulation of [33] or [34], wherein the tris(hydroxymethyl)aminomethane and/or a salt thereof is tris(hydroxymethyl)aminomethane-hydrochloride (Tris-HCl).
[36] The formulation of any one of [1] to [35], wherein the IL-31 antagonist is an antibody that inhibits IL-31 signaling.
[37] The formulation of [36], wherein the antibody that inhibits IL-31 signaling is an anti-IL-31 neutralizing antibody or an anti-IL-31RA neutralizing antibody.
[38] The formulation of [37], wherein the anti-IL-31RA neutralizing antibody is:
(1) an anti-IL-31RA antibody comprising an H chain variable region which comprises CDR1 of SEQ ID NO: 1, CDR2 of SEQ ID NO: 2, and CDR3 of SEQ ID NO: 3, and an L chain variable region which comprises CDR1 of SEQ ID NO: 4, CDR2 of SEQ ID NO: 5, and CDR3 of SEQ ID NO: 6;
(2) an anti-IL-31RA antibody comprising the H chain variable region of SEQ ID NO: 7 and the L chain variable region of SEQ ID NO: 8; or
(3) an anti-IL-31RA antibody comprising the H chain of SEQ ID NO: 9 and the L chain of SEQ ID NO: 10.
[39] The formulation of [37] or [38], wherein the anti-IL-31RA neutralizing antibody is an IgG antibody, preferably an IgG2 antibody.
[40] The formulation of any one of [37] to [39], wherein the anti-IL-31RA neutralizing antibody is Nemolizumab.
[41] The lyophilized formulation of any one of [37] to [40], which is a composition resulting from lyophilizing a solution in which the concentration of the anti-IL-31RA neutralizing antibody is 68 mg/mL.
[42] The lyophilized formulation of any one of [37] to [40], which is a composition resulting from lyophilizing a solution in which the concentration of the anti-IL-31RA neutralizing antibody is 50 mg/mL.
[43] The lyophilized formulation of any one of [37] to [40], which is a composition resulting from lyophilizing a solution in which the concentration of the anti-IL-31RA neutralizing antibody is 15 mg/mL.
[44] The lyophilized formulation of any one of [37] to [40], which is a composition resulting from lyophilizing a solution in which the concentration of the anti-IL-31RA neutralizing antibody is 7.5 mg/mL.
[45] The lyophilized formulation of any one of [37] to [41], which is a composition resulting from lyophilizing a solution in which the concentration of the Tris-HCl is 13.6 mmol/L.
[46] The lyophilized formulation of any one of [37] to [40] and [42], which is a composition resulting from lyophilizing a solution in which the concentration of the Tris-HCl is 10 mmol/L.
[47] The lyophilized formulation of any one of [37] to [40] and [43] to [44], which is a composition resulting from lyophilizing a solution in which the concentration of the Tris-HCl is 6 mmol/L.
[48] The lyophilized formulation of any one of [37] to [41] and [45], which is a composition resulting from lyophilizing a solution in which the concentration of the Arg-HCl is 102 mmol/L.
[49] The lyophilized formulation of any one of [37] to [40], [42], and [46], which is a composition resulting from lyophilizing a solution in which the concentration of the Arg-HCl is 75 mmol/L.
[50] The lyophilized formulation of any one of [37] to [40], [43] to [44], and [47], which is a composition resulting from lyophilizing a solution in which the concentration of the Arg-HCl is 45 mmol/L.
[51] The lyophilized formulation of any one of [37] to [41], [45], and [48], which is a composition resulting from lyophilizing a solution in which the concentration of the sucrose or trehalose is 170 mmol/L.
[52] The lyophilized formulation of any one of [37] to [40], [42], [46], and [49], which is a composition resulting from lyophilizing a solution in which the concentration of the sucrose or trehalose is 125 mmol/L.
[53] The lyophilized formulation of any one of [37] to [40], [43] to [44], [47], and [50], which is a composition resulting from lyophilizing a solution in which the concentration of the sucrose or trehalose is 75 mmol/L.
[54] The lyophilized formulation of any one of [37] to [41], [45], [48], and [51], which is a composition resulting from lyophilizing a solution in which the concentration of Poloxamer 188 or Polysorbate 20 is 0.34 mg/mL.
[55] The lyophilized formulation of any one of [37] to [40], [42], [46], [49], and [52], which is a composition resulting from lyophilizing a solution in which the concentration of Poloxamer 188 or Polysorbate 20 is 0.25 mg/mL.
[56] The lyophilized formulation of any one of [37] to [40], [43] to [44], [47], [50], and [53], which is a composition resulting from lyophilizing a solution in which the concentration of Poloxamer 188 or Polysorbate 20 is 0.15 mg/mL.
[57] The lyophilized formulation of any one of [37] to [42], [45] to [46], [48] to [49], [51] to [52], and [54] to [55], which is a composition resulting from lyophilizing a solution in which, between the arginine and the anti-IL-31RA neutralizing antibody, the molar ratio is 220:1 and/or the weight ratio is 0.3:1.
[58] The lyophilized formulation of any one of [37] to [40], [43], [47], [50], [53], and [56], which is a composition resulting from lyophilizing a solution in which, between the arginine and the anti-IL-31RA neutralizing antibody, the molar ratio is 440:1 and/or the weight ratio is 0.5:1.
[59] The lyophilized formulation of any one of [37] to [40], [44], [47], [50], [53], and [56], which is a composition resulting from lyophilizing a solution in which, between the arginine and the anti-IL-31RA neutralizing antibody, the molar ratio is 880:1 and/or the weight ratio is 1.0:1 to 1.1:1.
[60] The lyophilized formulation of any one of [37] to [42], [45] to [46], [48] to [49], [51] to [52], [54] to [55], and [57], which is a composition resulting from lyophilizing a solution in which, between the sucrose and the anti-IL-31RA neutralizing antibody, the molar ratio is 370:1 and/or the weight ratio is 0.8:1 to 0.9:1.
[61] The lyophilized formulation of any one of [37] to [40], [43], [47], [50], [53], [56], and [58], which is a composition resulting from lyophilizing a solution in which, between the sucrose and the anti-IL-31RA neutralizing antibody, the molar ratio is 740:1 and/or the weight ratio is 1.7:1.
[62] The lyophilized formulation of any one of [37] to [40], [44], [47], [50], [53], [56], and [59], which is a composition resulting from lyophilizing a solution in which, between the sucrose and the anti-IL-31RA neutralizing antibody, the molar ratio is 1470:1 and/or the weight ratio is 3.4:1 to 3.5:1.
[63] The lyophilized formulation of any one of [25] to [62], wherein the pH when the formulation is reconstituted in water is 6.5 to 7.5.
[64] The lyophilized formulation of any one of [25] to [63], wherein the pH when the formulation is reconstituted in water is 7.
[65] The lyophilized formulation of any one of [25] to [40], which comprises, per container such as syringe:
   75 mg anti-IL-31RA neutralizing antibody;
   1.8 mg tris(hydroxymethyl)aminomethane;
   19 mg arginine;
   64 mg sucrose;
   0.4 mg Poloxamer 188.
[66] The lyophilized formulation of any one of [25] to [40], which comprises, per container such as syringe:
   75 mg anti-IL-31RA neutralizing antibody;
   0.24 mg tris(hydroxymethyl)aminomethane;
   23.6 mg L-arginine hydrochloride;
   63.9 mg sucrose;
   0.37 mg Poloxamer 188; and
   tris(hydroxymethyl)aminomethane-hydrochloride as a pH adjusting agent.
[67] The lyophilized formulation of any one of [25] to [40], which comprises, per container such as syringe:
   39 mg anti-IL-31RA neutralizing antibody;
   0.9 mg tris(hydroxymethyl)aminomethane;
   10 mg arginine;
   33 mg sucrose; and
   0.2 mg Poloxamer 188.
[68] The lyophilized formulation of any one of [25] to [40], which comprises, per container such as cartridge:
   36 mg anti-IL-31RA neutralizing antibody;
   0.9 mg tris(hydroxymethyl)aminomethane;
   9 mg arginine;
   31 mg sucrose; and
   0.2 mg Poloxamer 188.
[69] The lyophilized formulation of any one of [25] to [40], which comprises, per container such as vial:
   51 mg anti-IL-31RA neutralizing antibody;
   2.5 mg tris(hydroxymethyl)aminomethane;
   27 mg arginine;
   88 mg sucrose; and
   0.5 mg Poloxamer 188.
[70] The lyophilized formulation of any one of [25] to [40], which comprises, per container such as vial:
   30 mg anti-IL-31RA neutralizing antibody;
   3.0 mg tris(hydroxymethyl)aminomethane;
   32 mg arginine;
   104 mg sucrose; and
   0.6 mg Poloxamer 188.
[71] The lyophilized formulation of any one of [25] to [40], which comprises, per container such as vial:
   19 mg anti-IL-31RA neutralizing antibody;
   1.9 mg tris(hydroxymethyl)aminomethane;
   20 mg arginine;
   66 mg sucrose; and
   0.4 mg Poloxamer 188.
[72] The lyophilized formulation of any one of [25] to [40], which comprises, per container such as vial:
   14 mg anti-IL-31RA neutralizing antibody;
   1.3 mg tris(hydroxymethyl)aminomethane;
   15 mg arginine;
   47 mg sucrose; and
   0.3 mg Poloxamer 188.
[73] The lyophilized formulation of any one of [25] to [40], which is a composition resulting from lyophilizing a solution comprising:
   68 mg/mL anti-IL-31RA antibody which comprises the H chain of SEQ ID NO: 9 and the L chain of SEQ ID NO: 10;
   13.6 mmol/L tris(hydroxymethyl)aminomethane-hydrochloride (Tris-HCl) buffer;
   102 mmol/L arginine-hydrochloride (Arg-HCl);
   170 mmol/L sucrose; and
   0.34 mg/mL Poloxamer 188,
      wherein pH is 7 when the formulation is reconstituted in water.
[74] The lyophilized formulation of any one of [25] to [40], which is a composition resulting from lyophilizing a solution comprising:
   50 mg/mL anti-IL-31RA antibody which comprises the H chain of SEQ ID NO: 9 and the L chain of SEQ ID NO: 10;
   10 mmol/L tris(hydroxymethyl)aminomethane-hydrochloride (Tris-HCl) buffer;
   75 mmol/L arginine-hydrochloride (Arg-HCl);
   125 mmol/L sucrose; and
   0.25 mg/mL Poloxamer 188,
      wherein pH is 7 when the formulation is reconstituted in water.
[75] The lyophilized formulation of any one of [25] to [40], which is a composition resulting from lyophilizing a solution comprising:
   15 mg/mL anti-IL-31RA antibody which comprises the H chain of SEQ ID NO: 9 and the L chain of SEQ ID NO: 10;
   6 mmol/L tris(hydroxymethyl)aminomethane-hydrochloride (Tris-HCl) buffer;
   45 mmol/L arginine-hydrochloride (Arg-HCl);
   75 mmol/L sucrose; and
   0.15 mg/mL Poloxamer 188,
      wherein pH is 7 when the formulation is reconstituted in water.
[76] The lyophilized formulation of any one of [25] to [40], which is a composition resulting from lyophilizing a solution comprising:
   7.5 mg/mL anti-IL-31RA antibody which comprises the H chain of SEQ ID NO: 9 and the L chain of SEQ ID NO: 10;
   6 mmol/L tris(hydroxymethyl)aminomethane-hydrochloride (Tris-HCl) buffer;
   45 mmol/L arginine-hydrochloride (Arg-HCl);
   75 mmol/L sucrose; and
   0.15 mg/mL Poloxamer 188,
      wherein pH is 7 when the formulation is reconstituted in water.
[77] A lyophilized formulation comprising an IL-31 antagonist as an active ingredient, the lyophilized formulation further comprising Tris buffer, arginine, sucrose or trehalose, and Poloxamer 188 or Polysorbate 20, wherein, when the formulation is reconstituted in water:
   1 to 200 mg/mL IL-31 antagonist;
   1 to 200 mmol/L Tris buffer;
   4.5 to 1500 mmol/L arginine or a salt thereof;
   7.5 to 2500 mmol/L sucrose or trehalose; and
   0.01 to 5 mg/mL Poloxamer 188 or Polysorbate 20
      are comprised, and pH is 6 to 8.
[78] A lyophilized formulation comprising an IL-31 antagonist as an active ingredient, the lyophilized formulation further comprising Tris buffer, arginine, sucrose or trehalose, and Poloxamer 188 or Polysorbate 20, wherein, when the formulation is reconstituted in water:
   20 to 100 mg/mL IL-31 antagonist;
   10 to 20 mmol/L Tris buffer;
   75 to 150 mmol/L arginine or a salt thereof;
   125 to 250 mmol/L sucrose or trehalose; and
   0.25 to 0.50 mg/mL Poloxamer 188 or Polysorbate 20
      are comprised, and pH is 6 to 8.
[79] The lyophilized formulation of [77] or [78], wherein the molar ratio of the arginine and/or salt thereof to the IL-31 antagonist is 220:1 to 1100:1, and/or the weight ratio of the arginine to the IL-31 antagonist is 0.3:1 to 1.3:1.
[80] The lyophilized formulation of any one of [77] to [79], wherein, between the sucrose or trehalose and the IL-31 antagonist, the molar ratio is 370:1 to 1840:1 and/or the weight ratio is 0.8:1 to 4.3:1.
[81] The lyophilized formulation of any one of [77] to [80], wherein the arginine and/or salt thereof is arginine-hydrochloride (Arg-HCl), arginine-aspartate, or arginine-glutamate.
[82] The lyophilized formulation of any one of [77] to [81], wherein the arginine and/or salt thereof is arginine-hydrochloride (Arg-HCl).
[83] The lyophilized formulation of any one of [77] to [82], wherein the Tris buffer is tris(hydroxymethyl)aminomethane and/or a salt thereof.
[84] The lyophilized formulation of [83], wherein the tris(hydroxymethyl)aminomethane and/or a salt thereof is tris(hydroxymethyl)aminomethane-hydrochloride (Tris-HCl), tris(hydroxymethyl)aminomethane-aspartate, tris(hydroxymethyl)aminomethane-glutamate, or tris(hydroxymethyl)aminomethane-acetate.
[85] The lyophilized formulation of [77] or [84], wherein the tris(hydroxymethyl)aminomethane and/or a salt thereof is tris(hydroxymethyl)aminomethane-hydrochloride (Tris-HCl).
[86] The lyophilized formulation of any one of [77] to [85], wherein the IL-31 antagonist is an antibody that inhibits IL-31 signaling.
[87] The lyophilized formulation of [86], wherein the antibody that inhibits IL-31 signaling is an anti-IL-31 neutralizing antibody or an anti-IL-31RA neutralizing antibody.
[88] The lyophilized formulation of [87], wherein the anti-IL-31RA neutralizing antibody is:
   (1) an anti-IL-31RA antibody comprising an H chain variable region which comprises CDR1 of SEQ ID NO: 1, CDR2 of SEQ ID NO: 2, and CDR3 of SEQ ID NO: 3, and an L chain variable region which comprises CDR1 of SEQ ID NO: 4, CDR2 of SEQ ID NO: 5, and CDR3 of SEQ ID NO: 6;
   (2) an anti-IL-31RA antibody comprising the H chain variable region of SEQ ID NO: 7 and the L chain variable region of SEQ ID NO: 8; or
   (3) an anti-IL-31RA antibody comprising the H chain of SEQ ID NO: 9 and the L chain of SEQ ID NO: 10.
[89] The lyophilized formulation of [87] or [88], wherein the anti-IL-31RA neutralizing antibody is an IgG antibody, preferably an IgG2 antibody.
[90] The lyophilized formulation of any one of [87] to [89], wherein the anti-IL-31RA neutralizing antibody is Nemolizumab.
[91] The lyophilized formulation of any one of [87] to [90], wherein the concentration of the anti-IL-31RA neutralizing antibody when the formulation is reconstituted in water is 100 mg/mL.
[92] The lyophilized formulation of any one of [87] to [90], wherein the concentration of the anti-IL-31RA neutralizing antibody when the formulation is reconstituted in water is 61.5 mg/mL.
[93] The lyophilized formulation of any one of [87] to [90], wherein the concentration of the anti-IL-31RA neutralizing antibody when the formulation is reconstituted in water is 50 mg/mL.
[94] The lyophilized formulation of any one of [87] to [90], wherein the concentration of the anti-IL-31RA neutralizing antibody when the formulation is reconstituted in water is 25 mg/mL.
[94] The lyophilized formulation of any one of [87] to [91], wherein the concentration of the Tris-HCl when the formulation is reconstituted in water is 20 mmol/L.
[96] The lyophilized formulation of any one of [87] to and wherein the concentration of the Tris-HCl when the formulation is reconstituted in water is 12.3 mmol/L.
[97] The lyophilized formulation of any one of [87] to [90] and [93] to [94], wherein the concentration of the Tris-HCl when the formulation is reconstituted in water is 20 mmol/L.
[98] The lyophilized formulation of any one of [87] to [91] and [95], wherein the concentration of the Arg-HCl when the formulation is reconstituted in water is 150 mmol/L.
[99] The lyophilized formulation of any one of [87] to [90], [92], and [96], wherein the concentration of the Arg-HCl when the formulation is reconstituted in water is 92 mmol/L.
[100] The lyophilized formulation of any one of [87] to [90], [93] to [94], and [97], wherein the concentration of the Arg-HCl when the formulation is reconstituted in water is 150 mmol/L.
[101] The lyophilized formulation of any one of [87] to [91], [95], and [98], wherein the concentration of the sucrose or trehalose when the formulation is reconstituted in water is 250 mmol/L.
[102] The lyophilized formulation of any one of [87] to [90], [92], [96], and [99], wherein the concentration of the sucrose or trehalose when the formulation is reconstituted in water is 154 mmol/L.
[103] The lyophilized formulation of any one of [87] to [90], [93] to [94], [97], and [100], wherein the concentration of the sucrose or trehalose when the formulation is reconstituted in water is 250 mmol/L.
[104] The lyophilized formulation of any one of [87] to [91], [95], [98], and [101], wherein the concentration of Poloxamer 188 or Polysorbate 20 when the formulation is reconstituted in water is 0.50 mg/mL.
[105] The lyophilized formulation of any one of [87] to [90], [92], [96], [99], and [102], wherein the concentration of Poloxamer 188 or Polysorbate 20 when the formulation is reconstituted in water is 0.31 mg/mL.
[106] The lyophilized formulation of any one of [87] to [90], [93] to [94], [97], [100], and [103], wherein the concentration of Poloxamer 188 or Polysorbate 20 when the formulation is reconstituted in water is 0.50 mg/mL.
[107] The lyophilized formulation of any one of [87] to [91], [95], [98], [101], and to [105], wherein, between the arginine and the anti-IL-31RA neutralizing antibody, the molar ratio is 220:1 and/or the weight ratio is 0.3:1.
[108] The lyophilized formulation of any one of [87] to [90], [93], [97], [100], [103], and [106], wherein, between the arginine and the anti-IL-31RA neutralizing antibody, the molar ratio is 440:1 and/or the weight ratio is 0.5:1.
[109] The lyophilized formulation of any one of [87] to [90], [94], [97], [100], [103], and [106], wherein, between the arginine and the anti-IL-31RA neutralizing antibody, the molar ratio is 880:1 and/or the weight ratio is 1.0:1 to 1.1:1.
[110] The lyophilized formulation of any one of [87] to [91], [95], [98], [101], [104] to [105], and [107], wherein, between the sucrose and the anti-IL-31RA neutralizing antibody, the molar ratio is 370:1 and/or the weight ratio is 0.8:1 to 0.9:1.
[111] The lyophilized formulation of any one of [87] to [90], [93], [97], [100], [103], [106], and [108], wherein, between the sucrose and the anti-IL-31RA neutralizing antibody, the molar ratio is 740:1 and/or the weight ratio is 1.7:1.
[112] The lyophilized formulation of any one of [87] to [90], [94], [97], [100], [103], and [109], wherein, between the sucrose and the anti-IL-31RA neutralizing antibody, the molar ratio is 1470:1 and/or the weight ratio is 3.4:1 to 3.5:1.
[113] The lyophilized formulation of any one of [77] to [112], wherein the pH when the formulation is reconstituted in water is 6.5 to 7.5.
[114] The lyophilized formulation of any one of [77] to [113], wherein the pH when the formulation is reconstituted in water is 6.7 to 7.3.
[115] The lyophilized formulation of any one of [77] to [114], wherein the pH when the formulation is reconstituted in water is 7.
[116] The lyophilized formulation of any one of [87] to [91], [95], [98], [101], [104], [107], and [110], wherein the lyophilized formulation is a pharmaceutical composition comprising as an active ingredient an anti-IL-31RA antibody which comprises the H chain of SEQ ID NO: 9 and the L chain of SEQ ID NO: 10, and is prepared by freeze-drying a solution comprising a buffer, a stabilizer, an excipient, and a nonionic surfactant, and wherein the following concentrations are made when the lyophilized formulation is reconstituted in water:
   the anti-IL-31RA neutralizing antibody at 100 mg/mL;
   tris(hydroxymethyl)aminomethane-hydrochloride (Tris-HCl) buffer at 20 mmol/L;
   arginine-hydrochloride (Arg-HCl) at 150 mmol/L;
   sucrose at 250 mmol/L; and
   Poloxamer 188 at 0.50 mg/mL.
[117] The lyophilized formulation of any one of [87] to [90], [92], [96], [99], [102], [105], [107], and [110], wherein the lyophilized formulation is a pharmaceutical composition comprising as an active ingredient an anti-IL-31RA antibody which comprises the H chain of SEQ ID NO: 9 and the L chain of SEQ ID NO: 10, and is prepared by freeze-drying a solution comprising a buffer, a stabilizer, an excipient, and a nonionic surfactant, and the following concentrations are made when the lyophilized formulation is reconstituted in water:
   the anti-IL-31RA neutralizing antibody at 61.5 mg/ml;
   tris(hydroxymethyl)aminomethane-hydrochloride (Tris-HCl) buffer at 12.3 mmol/L;
   arginine-hydrochloride (Arg-HCl) at 92 mmol/L;
   sucrose at 154 mmol/L; and
   Poloxamer 188 at 0.31 mg/mL.
[118] The lyophilized formulation of any one of [87] to [90], [93], [97], [100], [103], [106], [108], and [111], wherein the lyophilized formulation is a pharmaceutical composition comprising as an active ingredient an anti-IL-31RA antibody which comprises the H chain of SEQ ID NO: 9 and the L chain of SEQ ID NO: 10, and is prepared by freeze-drying a solution comprising a buffer, a stabilizer, an excipient, and a nonionic surfactant, and wherein the following concentrations are made when the lyophilized formulation is reconstituted in water:
   the anti-IL-31RA neutralizing antibody at 50 mg/mL;
   tris(hydroxymethyl)aminomethane-hydrochloride (Tris-HCl) buffer at 20 mmol/L;
   arginine-hydrochloride (Arg-HCl) at 150 mmol/L;
   sucrose at 250 mmol/L; and
   Poloxamer 188 at 0.50 mg/mL
[119] The lyophilized formulation of any one of [87] to [90], [94], [97], [100], [103], [109], and [112], wherein the lyophilized formulation is a pharmaceutical composition comprising as an active ingredient an anti-IL-31RA antibody which comprises the H chain of SEQ ID NO: 9 and the L chain of SEQ ID NO: 10, and is prepared by freeze-drying a solution comprising a buffer, a stabilizer, an excipient, and a nonionic surfactant, and wherein the following concentrations are made when the lyophilized formulation is reconstituted in water:
   the anti-IL-31RA neutralizing antibody at 25 mg/mL;
   tris(hydroxymethyl)aminomethane-hydrochloride (Tris-HCl) buffer at 20 mmol/L;
   arginine-hydrochloride (Arg-HCl) at 150 mmol/L;
   sucrose at 250 mmol/L; and
   Poloxamer 188 at 0.50 mg/mL.
[120] A lyophilized formulation comprising an IL-31 antagonist as an active ingredient, which is a composition resulting from lyophilizing a solution comprising:
   1 to 200 mg/mL IL-31 antagonist;
   1 to 200 mmol/L Tris buffer;
   4.5 to 1500 mmol/L arginine or a salt thereof;
   7.5 to 2500 mmol/L sucrose or trehalose; and
   0.01 to 5 mg/mL Poloxamer 188 or Polysorbate 20,
wherein, when the formulation is reconstituted in water:
   1 to 200 mg/mL IL-31 antagonist;
   1 to 200 mmol/L Tris buffer;
   4.5 to 1500 mmol/L arginine or a salt thereof;
   7.5 to 2500 mmol/L sucrose or trehalose; and
   0.01 to 5 mg/mL Poloxamer 188 or Polysorbate 20
      are comprised, and pH is 6 to 8.
[121] A lyophilized formulation comprising an IL-31 antagonist as an active ingredient, which is a composition resulting from lyophilizing a solution comprising:
   6 to 100 mg/mL IL-31 antagonist;
   6 to 20 mmol/L Tris buffer;
   45 to 150 mmol/L arginine or a salt thereof;
   75 to 250 mmol/L sucrose or trehalose; and
   0.15 to 0.50 mg/mL Poloxamer 188 or Polysorbate 20,
wherein, when the formulation is reconstituted in water:
   20 to 100 mg/mL IL-31 antagonist;
   10 to 20 mmol/L Tris buffer;
   75 to 150 mmol/L arginine or a salt thereof;
   125 to 250 mmol/L sucrose or trehalose; and
   0.25 to 0.50 mg/mL Poloxamer 188 or Polysorbate 20
      are comprised, and pH is 6 to 8.
[122] The lyophilized formulation of [120] or [121], wherein the molar ratio of the arginine and/or salt thereof to the IL-31 antagonist is 220:1 to 1100:1.
[123] The lyophilized formulation of any one of [120] to [122], wherein the molar ratio of the sucrose or trehalose to the IL-31 antagonist is 370:1 to 1840:1.
[124] The lyophilized formulation of any one of [120] to [123], wherein the arginine and/or salt thereof is arginine-hydrochloride (Arg-HCl), arginine-aspartate, or arginine-glutamate.
[125] The lyophilized formulation of any one of [120] to [124], wherein the arginine and/or salt thereof is arginine-hydrochloride (Arg-HCl).
[126] The lyophilized formulation of any one of [120] to [125], wherein the Tris buffer is tris(hydroxymethyl)aminomethane and/or a salt thereof.
[127] The lyophilized formulation of any one of [120] to [126], wherein the tris(hydroxymethyl)aminomethane and/or a salt thereof is tris(hydroxymethyl)aminomethane-hydrochloride (Tris-HCl), tris(hydroxymethyl)aminomethane-aspartate, tris(hydroxymethyl)aminomethane-glutamate, or tris(hydroxymethyl)aminomethane-acetate.
[128] The lyophilized formulation of any one of [120] or [127], wherein the tris(hydroxymethyl)aminomethane and/or a salt thereof is tris(hydroxymethyl)aminomethane-hydrochloride (Tris-HCl).
[129] The lyophilized formulation of any one of [120] to [128], wherein the IL-31 antagonist is an antibody that inhibits IL-31 signaling.
[130] The lyophilized formulation of [129], wherein the antibody that inhibits IL-31 signaling is an anti-IL-31 neutralizing antibody or an anti-IL-31RA neutralizing antibody.
[131] The lyophilized formulation of [130], wherein the anti-IL-31RA neutralizing antibody is:
(1) an anti-IL-31RA antibody comprising an H chain variable region which comprises CDR1 of SEQ ID NO: 1, CDR2 of SEQ ID NO: 2, and CDR3 of SEQ ID NO: 3, and an L chain variable region which comprises CDR1 of SEQ ID NO: 4, CDR2 of SEQ ID NO: 5, and CDR3 of SEQ ID NO: 6;
(2) an anti-IL-31RA antibody comprising the H chain variable region of SEQ ID NO: 7 and the L chain variable region of SEQ ID NO: 8; or
(3) an anti-IL-31RA antibody comprising the H chain of SEQ ID NO: 9 and the L chain of SEQ ID NO: 10.
[132] The lyophilized formulation of [130] or [131], wherein the anti-IL-31RA neutralizing antibody is an IgG antibody, preferably an IgG2 antibody.
[133] The lyophilized formulation of any one of [130] to [132], wherein the anti-IL-31RA neutralizing antibody is Nemolizumab.
[134] The formulation of any one of [1] to [133], for preventing and/or treating an IL-31-associated disorder or a symptom accompanying with the disorder.
[135] The formulation of [134], wherein the IL-31-associated disorder is an inflammatory disease related to IL-31 signaling.
[136] The formulation of [135], wherein the inflammatory disease is selected from the group consisting of atopic dermatitis, dialysis-induced pruritus, and prurigo nodularis.
[137] The formulation of any one of [1] to [136], wherein the IL-31 antagonist is subcutaneously administered.
[138] A method for stabilizing an antibody in an antibody-containing formulation, comprising preparing an antibody-containing solution which comprises arginine and/or a salt thereof, and/or sucrose and/or trehalose, wherein, in the solution, the molar ratio of the arginine and/or salt thereof to the antibody is 220:1 to 1100:1 and the molar ratio of the sucrose or trehalose to the antibody is 370:1 to 1840:1.
[139] The method of [138], wherein the antibody-containing solution further comprises 6 to 20 mmol/L tris(hydroxymethyl)aminomethane-hydrochloride (Tris-HCl) buffer and 0.15 to 0.50 mg/mL Poloxamer 188.
[140] The method of [138] or [139], further comprising preparing an antibody-containing lyophilized formulation by freeze-drying the antibody-containing solution.
[141] Use of arginine and/or a salt thereof, and/or sucrose and/or trehalose for stabilizing an antibody in an antibody-containing formulation, comprising preparing an antibody-containing solution which comprises arginine and/or a salt thereof, and/or sucrose and/or trehalose, wherein, in the solution, the molar ratio of the arginine and/or salt thereof to the antibody is 220:1 to 1100:1 and the molar ratio of the sucrose or trehalose to the antibody is 370:1 to 1840:1.
[142] The use of [141], wherein the antibody-containing solution further comprises 6 to 20 mmol/L tris(hydroxymethyl)aminomethane-hydrochloride (Tris-HCl) buffer and 0.15 to 0.50 mg/mL Poloxamer 188.
[143] The use of [141] or [142], further comprising preparing an antibody-containing lyophilized formulation by freeze-drying the antibody-containing solution.
[144] A method for suppressing antibody aggregation (aggregate formation) in an antibody-containing formulation, comprising preparing an antibody-containing solution which comprises arginine and/or a salt thereof, and/or sucrose and/or trehalose, wherein in the solution the molar ratio of the arginine and/or salt thereof to the antibody is 220:1 to 1100:1 and the molar ratio of the sucrose or trehalose to the antibody is 370:1 to 1840:1.
[145] The method of [144], wherein the antibody-containing solution further comprises 6 to 20 mmol/L tris(hydroxymethyl)aminomethane-hydrochloride (Tris-HCl) buffer and 0.15 to 0.50 mg/mL Poloxamer 188.
[146] The method of [144] or [145], further comprising preparing an antibody-containing lyophilized formulation by freeze-drying the antibody-containing solution.
[147] Use of arginine and/or a salt thereof, and/or sucrose and/or trehalose for suppressing antibody aggregation (aggregate formation) in an antibody-containing formulation, comprising preparing an antibody-containing solution which comprises arginine and/or a salt thereof, and/or sucrose and/or trehalose, wherein, in the solution, the molar ratio of the arginine and/or salt thereof to the antibody is 220:1 to 1100:1 and the molar ratio of the sucrose or trehalose to the antibody is 370:1 to 1840:1.
[148] The use of [147], wherein the antibody-containing solution further comprises 6 to 20 mmol/L tris(hydroxymethyl)aminomethane-hydrochloride (Tris-HCl) buffer and 0.15 to 0.50 mg/mL Poloxamer 188.
[149] The use of [147] or [148], further comprising preparing an antibody-containing lyophilized formulation by freeze-drying the antibody-containing solution.
[150] A method for reducing components with charge heterogeneity in an antibody-containing formulation, comprising preparing an antibody-containing solution which comprises arginine and/or a salt thereof, and/or sucrose and/or trehalose, wherein, in the solution, the molar ratio of the arginine and/or salt thereof to the antibody is 220:1 to 1100:1 and the molar ratio of the sucrose or trehalose to the antibody is 370:1 to 1840:1.
[151] The method of [150], wherein the antibody-containing solution further comprises 6 to 20 mmol/L tris(hydroxymethyl)aminomethane-hydrochloride (Tris-HCl) buffer and 0.15 to 0.50 mg/mL Poloxamer 188.
[152] The method of [150] or [151], further comprising preparing an antibody-containing lyophilized formulation by freeze-drying the antibody-containing solution.
[153] Use of arginine and/or a salt thereof, and/or sucrose and/or trehalose for reducing components with charge heterogeneity in an antibody-containing formulation, comprising preparing an antibody-containing solution which comprises arginine and/or a salt thereof, and/or sucrose and/or trehalose, wherein, in the solution, the molar ratio of the arginine and/or salt thereof to the antibody is 220:1 to 1100:1 and the molar ratio of the sucrose or trehalose to the antibody is 370:1 to 1840:1.
[154] The use of [153], wherein the antibody-containing solution further comprises 6 to 20 mmol/L tris(hydroxymethyl)aminomethane-hydrochloride (Tris-HCl) buffer and 0.15 to 0.50 mg/mL Poloxamer 188.
[155] The use of [153] or [154], further comprising preparing an antibody-containing lyophilized formulation by freeze-drying the antibody-containing solution.
[156] A method for preventing and/or treating an IL-31-associated disorder, comprising administering an IL-31 antagonist to a subject affected with or at a risk of being affected with the IL-31-associated disorder, the method comprising:
preparing a lyophilized formulation by freeze-drying a solution comprising 6 to 100 mg/mL IL-31 antagonist, 45 to 150 mmol/L arginine-hydrochloride (Arg-HCl), and 75 to 250 mmol/L sucrose or trehalose;
preparing a reconstituted solution by reconstituting the lyophilized formulation in water; and administering the reconstituted solution to the subject.
[157] Use of an IL-31 antagonist in the manufacture of a medicament for preventing and/or treating an IL-31-associated disorder, characterized in that a lyophilized formulation is prepared by freeze-drying a solution comprising 6 to 100 mg/mL IL-31 antagonist, 45 to 150 mmol/L arginine-hydrochloride (Arg-HCl), and 75 to 250 mmol/L sucrose or trehalose.
[158] An IL-31 antagonist for use in preventing and/or treating an IL-31-associated disorder, characterized in that a lyophilized formulation resulting from lyophilizing a solution comprising 6 to 100 mg/mL IL-31 antagonist, 45 to 150 mmol/L arginine-hydrochloride (Arg-HCl), and 75 to 250 mmol/L sucrose or trehalose is reconstituted in water prior to use.
[159] The method of [156], the use of [157], or the IL-31 antagonist of [158], wherein the solution before lyophilization further comprises 6 to 20 mmol/L tris(hydroxymethyl)aminomethane-hydrochloride (Tris-HCl) buffer and 0.15 to 0.50 mg/mL Poloxamer 188.
[160] A formulation for injection or a kit comprising (i) a container, (ii) the lyophilized formulation of any one of [1] to [12] and [25] to [137], and (iii) water for injection for reconstituting the lyophilized formulation.
[161] A formulation for injection comprising (i) a container, (ii) a lyophilized formulation in the container, the lyophilized formulation resulting from lyophilizing a solution comprising 6 to 100 mg/mL anti-IL-31RA neutralizing antibody, 45 to 150 mmol/L arginine-hydrochloride (Arg-HCl), and 75 to 250 mmol/L sucrose or trehalose, and (iii) optionally, water for injection for reconstituting the lyophilized formulation.
[162] The formulation for injection of [161], wherein the solution before lyophilization further comprises 6 to 20 mmol/L tris(hydroxymethyl)aminomethane-hydrochloride (Tris-HCl) buffer and 0.15 to 0.50 mg/mL Poloxamer 188.
[163] A formulation for injection comprising (i) a container, (ii) a lyophilized formulation in the container, the lyophilized formulation resulting from lyophilizing a solution comprising an effective amount of anti-IL-31RA neutralizing antibody, and 220 to 1100 mole of arginine-hydrochloride (Arg-HCl) and 370 to 1840 mole of sucrose or trehalose per 1 mole of the antibody, and (iii) optionally, water for injection for reconstituting the lyophilized formulation.
[164] The formulation for injection or kit of any one of [160] to [163], wherein the lyophilized formulation and the water for injection are enclosed in separate compartments in the container.
[165] The formulation for injection or kit of any one of [160] to [164], wherein the lyophilized formulation is reconstituted using the water for injection within the container when used.

### DESCRIPTION OF EMBODIMENTS

Preferred non-limiting embodiments of the present disclosure will be hereinafter described.

All the embodiments set forth in the Examples below are described with the intention that they are naturally construed as being equivalently described in the “Description of Embodiments” of the present specification, without being restricted by any patent practice, conventions, or law that may interpret the contents of the Examples in a limiting manner, in a country where it is intended that the protection of the present patent application be sought.

For descriptions for numerical values in the present disclosure, a numerical value at the smallest digit (for example, ones place) may include values in which one place smaller than the smallest digit (for example, when the smallest digit is ones place, first decimal place) is rounded off. For example, it is intended that the numerical value “5” comprises numerical values comprised in the rages of 4.5 to 5.4.

In a non-limiting embodiment, the present disclosure relates to a lyophilized formulation comprising an IL-31 antagonist as an active ingredient, the lyophilized formulation comprising arginine and/or salts thereof, and sucrose and/or trehalose. In another non-limiting embodiment, the present disclosure relates to a solution formulation comprising an IL-31 antagonist as an active ingredient, the solution formulation comprising arginine and/or salts thereof. Arginine is preferably L-arginine. In one embodiment, arginine and/or a salt thereof is arginine-hydrochloride, L-arginine-hydrochloride, arginine-aspartate, L-arginine-aspartate, arginine-glutamate, or L-arginine-glutamate. Those skilled in the art will understand that, when the weight (e.g., mg) of arginine is referred to, the weight may be the weight of free arginine, or the weight of arginine contained in a salt comprising arginine. Furthermore, in the present disclosure, the weight or concentration of arginine may be a sum of the weight or concentration of free arginine and the weight or concentration of arginine contained in a salt comprising arginine. In one embodiment, the formulation of the present disclosure further comprises Tris buffer as a buffer. In one embodiment, Tris buffer is tris(hydroxymethyl)aminomethane and/or a salt thereof, for example tris(hydroxymethyl)aminomethane-hydrochloride (Tris-HCl), tris(hydroxymethyl)aminomethane-aspartate, tris(hydroxymethyl)aminomethane-glutamate, or tris(hydroxymethyl)aminomethane-acetate. In one embodiment, the formulation of the present disclosure further comprises Poloxamer 188 or polysorbate as a nonionic surfactant. Poloxamer 188 may be called “polyoxyethylene (160) polyoxypropylene (30) glycol” in Standards of Pharmacopoeia of Japan. In one embodiment, polysorbate is Polysorbate 20 or Polysorbate 80. In one embodiment, pH of a reconstituted solution after reconstituting the lyophilized formulation of the present disclosure in water is 6 to 8, for example 6.5 to 7.5, for example 6.7 to 7.3, and for example 7. In one embodiment, pH of the solution formulation of the present disclosure is 6 to 8, for example 6.5 to 7.5, and for example 7. Meanwhile, tris(hydroxymethyl)aminomethane may be called “Trometamol” in Standards of Pharmacopoeia of Japan. Those skilled in the art will understand that, when the weight (e.g., mg) of tris(hydroxymethyl)aminomethane is mentioned, the weight may be the weight of free tris(hydroxymethyl)aminomethane or the weight of tris(hydroxymethyl)aminomethane contained in a salt comprising tris(hydroxymethyl)aminomethane. Furthermore, in the present disclosure, the weight or concentration of tris(hydroxymethyl)aminomethane may be a sum of the weight or concentration of free tris(hydroxymethyl)aminomethane and the weight or concentration of tris(hydroxymethyl)aminomethane included in salts comprising tris(hydroxymethyl)aminomethane.

Amount and concentration of IL-31 antagonist comprised in the formulations of the present disclosure are not particularly limited, and can be adjusted appropriately depending on a subject to be administrated, for example, whether the formulations are for adults or for children, or whether the formulations are for prevention or for treatment, or depending on type, severity, and such of disorders or symptoms to be prevented or treated. Therefore, molar ratio and weight ratio of IL-31 antagonist to another component comprised in the formulation of the present disclosure can be various values. In a non-limiting embodiment, the molar ratio of arginine and/or a salt thereof to IL-31 antagonist is 3:1 to 220500:1, for example 22:1 to 6600:1, 33:1 to 5500:1, 44:1 to 4400:1, 55:1 to 3300:1, 110:1 to 2200:1, 220:1 to 1100:1, 220:1 to 880:1, 220:1 to 440:1, or 440:1 to 880:1. In a non-limiting embodiment, the weight ratio of arginine to IL-31 antagonist is 0.004:1 to 261:1, for example 0.03:1 to 9.6:1, 0.1:1 to 4.8:1, 0.2:1 to 3.2:1, 0.3:1 to 1.6:1, 0.3:1 to 1.3:1, 0.3:1 to 1.1:1, 0.5:1 to 1.1:1, or 0.3:1 to 0.5:1. In a non-limiting embodiment, the molar ratio of sucrose or trehalose to IL-31 antagonist is 6:1 to 367500:1, for example 75:1 to 7360:1, 100:1 to 5520:1, 150:1 to 3680:1, 300:1 to 1840:1, 370:1 to 1840:1, 370:1 to 1470:1, 740:1 to 1470:1, or 370:1 to 740:1. In a non-limiting embodiment, the weight ratio of sucrose or trehalose to IL-31 antagonist is 0.013:1 to 856:1, for example 0.18:1 to 16.8:1, 0.23:1 to 12.6:1, 0.35:1 to 8.4:1, 0.7:1 to 4.2:1, 0.8:1 to 4.3:1, 0.8:1 to 3.5:1, 1.7:1 to 3.5:1, or 0.8:1 to 1.7:1. Meanwhile, sucrose may be called “Seisei Hakutou (purified sucrose)” in Standards of Pharmacopoeia of Japan.

In a non-limiting embodiment, the concentration of an IL-31 antagonist comprised in the formulation of the present disclosure in a solution state (i.e., concentration in a solution formulation, concentration in a solution before lyophilization of a lyophilized solution, or concentration in a solution after redissolution of a lyophilized formulation) is 1 mg/mL to 200 mg/mL, for example 2 mg/mL to 200 mg/mL, 3 mg/mL to 200 mg/mL, 3 mg/mL to 150 mg/mL, 6 mg/mL to 100 mg/mL, 10 mg/mL to 100 mg/mL, 15 mg/mL to 100 mg/mL, 30 mg/mL to 100 mg/mL, 50 mg/mL to 100 mg/mL, 6 mg/mL to 70 mg/mL, 10 mg/mL to 70 mg/mL, 15 mg/mL to 70 mg/mL, 30 mg/mL to 70 mg/mL, 50 mg/mL to 70 mg/mL, 6 mg/mL to 50 mg/mL, 10 mg/mL to 50 mg/mL, 30 mg/mL to 50 mg/mL, or 6 mg/mL to 30 mg/mL.

In one embodiment, the concentration of arginine and/or a salt thereof comprised in the formulation of the present disclosure in a solution state is 4.5 mmol/L to 1500 mmol/L, for example 9 mmol/L to 750 mmol/L, 15 mmol/L to 450 mmol/L, 22.5 mmol/L to 300 mmol/L, 45 mmol/L to 150 mmol/L, 75 mmol/L to 150 mmol/L, or 45 mmol/L to 75 mmol/L.

In one embodiment, the concentration of sucrose and/or trehalose comprised in the formulation of the present disclosure in a solution state is 7.5 mmol/L to 2500 mmol/L, for example 15 mmol/L to 1250 mmol/L, 25 mmol/L to 750 mmol/L, 37.5 mmol/L to 500 mmol/L, 75 mmol/L to 250 mmol/L, 125 mmol/L to 250 mmol/L, 75 mmol/L to 170 mmol/L, 125 mmol/L to 170 mmol/L, or 75 mmol/L to 125 mmol/L.

In one embodiment, the concentration of Tris buffer comprised in the formulation of the present disclosure in a solution state is 1 mmol/L to 200 mmol/L, for example 1 mmol/L to 100 mmol/L, 2 mmol/L to 60 mmol/L, 3 mmol/L to 40 mmol/L, 6 mmol/L to 20 mmol/L, 3 mmol/L to 15 mmol/L, 10 mmol/L to 20 mmol/L, 10 mmol/L to 15 mmol/L, 6 mmol/L to 15 mmol/L, or 6 mmol/L to 10 mmol/L.

In one embodiment, the concentration of Poloxamer188 or polysorbate comprised in the formulation of the present disclosure in a solution state is 0.015 mg/mL to 5 mg/mL, for example 0.03 mg/mL to 2.5 mg/mL, 0.05 mg/mL to 1.5 mg/mL, 0.075 mg/mL to 1 mg/mL, 0.15 mg/mL to 0.5 mg/mL, 0.15 mg/mL to 0.4 mg/mL, 0.25 mg/mL to 0.5 mg/mL, or 0.15 mg/mL to 0.25 mg/mL.

In one embodiment, the lyophilized formulation of the present disclosure is a composition resulting from lyophilizing a solution comprising:
1 mg/mL to 200 mg/mL IL-31 antagonist;
1 mmol/L to 200 mmol/L Tris buffer;
4.5 mmol/L to 1500 mmol/L arginine or a salt thereof;
7.5 mmol/L to 2500 mmol/L sucrose or trehalose; and
0.01 mg/mL to 5 mg/mL Poloxamer 188 or Polysorbate 20,
   wherein, when the formulation is reconstituted in water, pH is 6 to 8, for example 6.5 to 7.5, and for example 7.

In a particular embodiment, the lyophilized formulation of the present disclosure is a composition resulting from lyophilizing a solution comprising:
6 mg/mL to 100 mg/mL IL-31 antagonist;
6 mmol/L to 20 mmol/L Tris buffer;
45 mmol/L to 150 mmol/L arginine or a salt thereof;
75 mmol/L to 250 mmol/L sucrose or trehalose; and
0.15 mg/mL to 0.50 mg/mL Poloxamer 188 or Polysorbate 20,
   wherein, when the formulation is reconstituted in water, pH is 6 to 8, for example 6.5 to 7.5, and for example 7.

In a particular embodiment, the lyophilized formulation of the present disclosure is a composition resulting from lyophilizing a solution comprising, per vial, cartridge, or syringe:
1 mg to 800 mg of IL-31 antagonist;
0.1 mg to 40 mg of tris(hydroxymethyl)aminomethane;
0.8 mg to 400 mg of arginine;
3 mg to 1100 mg of sucrose or trehalose; and
0.01 mg to 7 mg of Poloxamer 188 or Polysorbate 20,
   wherein, when the formulation is reconstituted in water, pH is 6 to 8, for example 6.5 to 7.5, for example 7.

In a particular embodiment, the lyophilized formulation of the present disclosure is a composition resulting from lyophilizing a solution comprising, per vial, cartridge, or syringe:
10 mg to 80 mg of IL-31 antagonist;
0.8 mg to 4 mg of tris(hydroxymethyl)aminomethane;
8 mg to 40 mg of arginine;
30 mg to 110 mg of sucrose or trehalose; and
0.1 mg to 0.7 mg of Poloxamer 188 or Polysorbate 20,
   wherein, when the formulation is reconstituted in water, pH is 6 to 8, for example 6.5 to 7.5, for example 7.

In one embodiment, the lyophilized formulation of the present disclosure is a composition resulting from lyophilizing a solution comprising:
61 mg/mL to 75 mg/mL, for example 68 mg/mL anti-IL-31RA neutralizing antibody;
11 mmol/L to 16 mmol/L, for example 13.6 mmol/L Tris-HCl;
82 mmol/L to 122 mmol/L, for example 102 mmol/L Arg-HCl;
153 mmol/L to 187 mmol/L, for example 170 mmol/L sucrose or trehalose; and
0.17 mg/mL to 0.51 mg/mL, for example 0.34 mg/mL Poloxamer 188 or Polysorbate 20,
   wherein, when the formulation is reconstituted in water, pH is 6 to 8, for example 6.5 to 7.5, and for example 7.

In another embodiment, the lyophilized formulation of the present disclosure is a composition resulting from lyophilizing a solution comprising:
45 mg/mL to 55 mg/mL, for example 50 mg/mL anti-IL-31RA neutralizing antibody;
8 mmol/L to 12 mmol/L, for example 10 mmol/L Tris-HCl;
60 mmol/L to 90 mmol/L, for example 75 mmol/L Arg-HCl;
113 mmol/L to 138 mmol/L, for example 125 mmol/L sucrose or trehalose; and
0.13 mg/mL to 0.38 mg/mL, for example 0.25 mg/mL Poloxamer 188 or Polysorbate 20,
   wherein, when the formulation is reconstituted in water, pH is 6 to 8, for example 6.5 to 7.5, and for example 7.

In still another embodiment, the lyophilized formulation of the present disclosure is a composition resulting from lyophilizing a solution comprising:
14 mg/mL to 17 mg/mL, for example 15 mg/mL anti-IL-31RA neutralizing antibody;
5 mmol/L to 7 mmol/L, for example 6 mmol/L Tris-HCl;
36 mmol/L to 54 mmol/L, for example 45 mmol/L Arg-HCl;
68 mmol/L to 83 mmol/L, for example 75 mmol/L sucrose or trehalose; and
0.08 mg/mL to 0.23 mg/mL, for example 0.15 mg/mL Poloxamer 188 or Polysorbate 20,
   wherein, when the formulation is reconstituted in water, pH is 6 to 8, for example 6.5 to 7.5, and for example 7.

In a further embodiment, the lyophilized formulation of the present disclosure is a composition resulting from lyophilizing a solution comprising:
7 mg/mL to 8 mg/mL, for example 7.5 mg/mL anti-IL-31RA neutralizing antibody;
5 mmol/L to 7 mmol/L, for example 6 mmol/L Tris-HCl;
5 36 mmol/L to 54 mmol/L, for example 45 mmol/L Arg-HCl;
68 mmol/L to 83 mmol/L, for example 75 mmol/L sucrose or trehalose; and
0.08 mg/mL to 0.23 mg/mL, for example 0.15 mg/mL Poloxamer 188 or Polysorbate 20,
   wherein, when the formulation is reconstituted in water, pH is 6 to 8, for example 6.5 to 7.5, and for example 7.

The amount of each component contained in the lyophilized formulation of the present disclosure can also be represented as weight (mass) per container such as vial, cartridge, and syringe. A container may contain a single dose or multiple doses (for example, two doses, three doses, four doses, etc.) of the formulation. For example, a container contains a single dose of the formulation. Multiple containers may also be used for a single dose. The dose per administration may be appropriately determined according to the method of administration (e.g. subcutaneous administration), target disease (e.g. atopic dermatitis), purpose of use (e.g. for prevention or treatment), type of patient (e.g. adult or child), patient's conditions (e.g. severity of pruritus), and the like. In accordance with that, the amount of solvent such as water to be added to the container for reconstituting the lyophilized formulation can be appropriately determined. When the formulation is subcutaneously administered, the liquid volume for a single dose (in the case of the lyophilized formulation, the volume of liquid to be administered after reconstitution with water) may be, for example, 0.1 mL to 10 mL, 0.2 mL to 5 mL, 0.2 mL to 1.6 mL, 0.5 mL to 2 mL, 0.8 mL to 1.2 mL, or 0.9 mL to 1.1 mL, and for example, 1.0 mL, but is not limited thereto. It should be noted that those skilled in the art will naturally understand that a container may be overfilled with the formulation to ensure that an amount sufficient for administering a single dose or multiple doses of the IL-31 antagonist can be obtained from one container (such as vial, cartridge, or syringe), taking into account a loss of drug liquid when being administered.

In one embodiment, the lyophilized formulation of the present disclosure comprises, per container such as syringe:
68 mg to 83 mg, for example 75 mg of anti-IL-31RA neutralizing antibody (an anti-IL-31RA antibody comprising the H chain of SEQ ID NO: 9 and the L chain of SEQ ID NO: 10);
1.4 mg to 2.2 mg, for example 1.8 mg of tris(hydroxymethyl)aminomethane;
15 mg to 23 mg, for example 19 mg of arginine;
58 mg to 70 mg, for example 64 mg of sucrose; and
0.2 mg to 0.6 mg, for example 0.4 mg of Poloxamer 188.

In the above-mentioned embodiment, regarding the descriptions of numerical values, it is intended that a numerical value at the smallest digit (for example, ones place) includes values in which one place smaller than the smallest digit (for example, when the smallest digit is ones place, first decimal place) is rounded off. More specifically, the lyophilized formulation of the present disclosure can also be referred to as comprising, per container such as syringe:
75 mg (74.5 mg to 75.4 mg) of anti-IL-31RA neutralizing antibody (an anti-IL-31RA antibody comprising the H chain of SEQ ID NO: 9 and the L chain of SEQ ID NO: 10);
1.8 mg (1.75 mg to 1.84 mg) of tris(hydroxymethyl)aminomethane;
19 mg (18.5 mg to 19.4 mg) of arginine;
64 mg (63.5 mg to 64.4 mg) of sucrose; and
0.4 mg (0.35 mg to 0.44 mg) of Poloxamer 188.

Furthermore, in the above-mentioned embodiment, the weight of the above-mentioned tris(hydroxymethyl)aminomethane may be a sum of the weight of free tris(hydroxymethyl)aminomethane and the weight of tris(hydroxymethyl)aminomethane contained in tris(hydroxymethyl)aminomethane hydrochloride (which may be added as a pH adjuster). In addition, the above-mentioned arginine may be added as a salt containing arginine (e.g., arginine hydrochloride). Those skilled in the art will understand that when a salt containing arginine is added, the weight of the above-mentioned arginine is the weight of arginine contained in the salt.

Accordingly, the lyophilized formulation of the present disclosure can also be referred to as comprising, per container such as syringe:
75 mg of anti-IL-31RA neutralizing antibody (an anti-IL-31RA antibody comprising the H chain of SEQ ID NO: 9 and the L chain of SEQ ID NO: 10);
0.24 mg of tris(hydroxymethyl)aminomethane;
23.6 mg of L-arginine hydrochloride;
63.9 mg of sucrose;
0.37 mg of Poloxamer 188; and
tris(hydroxymethyl)aminomethane hydrochloride as a pH adjuster.

In another embodiment, the lyophilized formulation of the present disclosure comprises, per container such as syringe:
35 mg to 43 mg, for example 39 mg of anti-IL-31RA neutralizing antibody (an anti-IL-31RA antibody comprising the H chain of SEQ ID NO: 9 and the L chain of SEQ ID NO: 10);
0.7 mg to 1.1 mg, for example 0.9 mg of tris(hydroxymethyl)aminomethane;
8 mg to 12 mg, for example 10 mg of arginine;
30 mg to 36 mg, for example 33 mg of sucrose; and
0.1 mg to 0.3 mg, for example 0.2 mg of Poloxamer 188.

In the above-mentioned embodiment, regarding the descriptions of numerical values, it is intended that a numerical value at the smallest digit (for example, ones place) includes values in which one place smaller than the smallest digit (for example, when the smallest digit is ones place, first decimal place) is rounded off. More specifically, the lyophilized formulation of the present disclosure can also be referred to as comprising, per container such as syringe:
39 mg (38.5 mg to 39.4 mg) of anti-IL-31RA neutralizing antibody (an anti-IL-31RA antibody comprising the H chain of SEQ ID NO: 9 and the L chain of SEQ ID NO: 10);
0.9 mg (0.85 mg to 0.94 mg) of tris(hydroxymethyl)aminomethane;
10 mg (9.5 mg to 10.4 mg) of arginine;
33 mg (32.5 mg to 33.4 mg) of sucrose; and
0.2 mg (0.15 mg to 0.24 mg) of Poloxamer 188.

In a further embodiment, the lyophilized formulation of the present disclosure comprises, per container such as cartridge:
32 mg to 40 mg, for example 36 mg of anti-IL-31RA neutralizing antibody (an anti-IL-31RA antibody comprising the H chain of SEQ ID NO: 9 and the L chain of SEQ ID NO: 10);
0.7 mg to 1.1 mg, for example 0.9 mg of tris(hydroxymethyl)aminomethane;
7 mg to 11 mg, for example 9 mg of arginine;
28 mg to 34 mg, for example 31 mg of sucrose; and
0.1 mg to 0.3 mg, for example 0.2 mg of Poloxamer 188.

In the above-mentioned embodiment, regarding the descriptions of numerical values, it is intended that a numerical value at the smallest digit (for example, ones place) includes values in which one place smaller than the smallest digit (for example, when the smallest digit is ones place, first decimal place) is rounded off. More specifically, the lyophilized formulation of the present disclosure can also be referred to as comprising, per container such as cartridge:
36 mg (35.5 mg to 36.4 mg) of anti-IL-31RA neutralizing antibody (an anti-IL-31RA antibody comprising the H chain of SEQ ID NO: 9 and the L chain of SEQ ID NO: 10);
0.9 mg (0.85 mg to 0.94 mg) of tris(hydroxymethyl)aminomethane;
9 mg (8.5 mg to 9.4 mg) of arginine;
31 mg (30.5 mg to 31.4 mg) of sucrose; and
0.2 mg (0.15 mg to 0.24 mg) of Poloxamer 188.

In still another embodiment, the lyophilized formulation of the present disclosure comprises, per container such as vial:
46 mg to 56 mg, for example 51 mg of anti-IL-31RA neutralizing antibody (an anti-IL-31RA antibody comprising the H chain of SEQ ID NO: 9 and the L chain of SEQ ID NO: 10);
2.0 mg to 3.0 mg, for example 2.5 mg of tris(hydroxymethyl)aminomethane;
22 mg to 32 mg, for example 27 mg of arginine;
79 mg to 97 mg, for example 88 mg of sucrose; and
0.3 mg to 0.8 mg, for example 0.5 mg of Poloxamer 188.

In the above-mentioned embodiment, regarding the descriptions of numerical values, it is intended that a numerical value at the smallest digit (for example, ones place) includes values in which one place smaller than the smallest digit (for example, when the smallest digit is ones place, first decimal place) is rounded off. More specifically, the lyophilized formulation of the present disclosure can also be referred to as comprising, per container such as vial:
51 mg (50.5 mg to 51.4 mg) of anti-IL-31RA neutralizing antibody (an anti-IL-31RA antibody comprising the H chain of SEQ ID NO: 9 and the L chain of SEQ ID NO: 10);
2.5 mg (2.45 mg to 2.54 mg) of tris(hydroxymethyl)aminomethane;
27 mg (26.5 mg to 27.4 mg) of arginine;
88 mg (87.5 mg to 88.4 mg) of sucrose; and
0.5 mg (0.45 mg to 0.54 mg) of Poloxamer 188.

Furthermore, in another embodiment, the lyophilized formulation of the present disclosure comprises, per container such as vial:
27 mg to 33 mg, for example 30 mg of anti-IL-31RA neutralizing antibody (an anti-IL-31RA antibody comprising the H chain of SEQ ID NO: 9 and the L chain of SEQ ID NO: 10);
2.4 mg to 3.6 mg, for example 3.0 mg of tris(hydroxymethyl)aminomethane;
26 mg to 38 mg, for example 32 mg of arginine;
94 mg to 114 mg, for example 104 mg of sucrose; and
0.3 mg to 0.9 mg, for example 0.6 mg of Poloxamer 188.

In the above-mentioned embodiment, regarding the descriptions of numerical values, it is intended that a numerical value at the smallest digit (for example, ones place) includes values in which one place smaller than the smallest digit (for example, when the smallest digit is ones place, first decimal place) is rounded off. More specifically, the lyophilized formulation of the present disclosure can also be referred to as comprising, per container such as vial:
30 mg (29.5 mg to 30.4 mg) of anti-IL-31RA neutralizing antibody (an anti-IL-31RA antibody comprising the H chain of SEQ ID NO: 9 and the L chain of SEQ ID NO: 10);
3.0 mg (2.95 mg to 3.04 mg) of tris(hydroxymethyl)aminomethane;
32 mg (31.5 mg to 32.4 mg) of arginine;
104 mg (103.5 mg to 104.4 mg) of sucrose; and
0.6 mg (0.55 mg to 0.64 mg) of Poloxamer 188.

In a still another embodiment, the lyophilized formulation of the present disclosure comprises, per container such as vial:
17 mg to 21 mg, for example 19 mg of anti-IL-31RA neutralizing antibody (an anti-IL-31RA antibody comprising the H chain of SEQ ID NO: 9 and the L chain of SEQ ID NO: 10);
1.5 mg to 2.3 mg, for example 1.9 mg of tris(hydroxymethyl)aminomethane;
16 mg to 24 mg, for example 20 mg of arginine;
59 mg to 73 mg, for example 66 mg of sucrose; and
0.2 mg to 0.6 mg, for example 0.4 mg of Poloxamer 188.

In the above-mentioned embodiment, regarding the descriptions of numerical values, it is intended that a numerical value at the smallest digit (for example, ones place) includes values in which one place smaller than the smallest digit (for example, when the smallest digit is ones place, first decimal place) is rounded off. More specifically, the lyophilized formulation of the present disclosure can also be referred to as comprising, per container such as vial:
19 mg (18.5 mg to 19.4 mg) of anti-IL-31RA neutralizing antibody (an anti-IL-31RA antibody comprising the H chain of SEQ ID NO: 9 and the L chain of SEQ ID NO: 10);
1.9 mg (1.85 mg to 1.94 mg) of tris(hydroxymethyl)aminomethane;
20 mg (19.5 mg to 20.4 mg) of arginine;
66 mg (65.5 mg to 66.4 mg) of sucrose; and
0.4 mg (0.35 mg to 0.44 mg) of Poloxamer 188.

In a further embodiment, the lyophilized formulation of the present disclosure comprises, per container such as vial:
13 mg to 15 mg, for example 14 mg of anti-IL-31RA neutralizing antibody (an anti-IL-31RA antibody comprising the H chain of SEQ ID NO: 9 and the L chain of SEQ ID NO: 10);
1.0 mg to 1.6 mg, for example 1.3 mg of tris(hydroxymethyl)aminomethane;
12 mg to 18 mg, for example 15 mg of arginine;
42 mg to 52 mg, for example 47 mg of sucrose; and
0.2 mg to 0.5 mg, for example 0.3 mg of Poloxamer 188.

In the above-mentioned embodiment, regarding the descriptions of numerical values, it is intended that a numerical value at the smallest digit (for example, ones place) includes values in which one place smaller than the smallest digit (for example, when the smallest digit is ones place, first decimal place) is rounded off. More specifically, the lyophilized formulation of the present disclosure can also be referred to as comprising, per container such as vial:
14 mg (13.5 mg to 14.4 mg) of anti-IL-31RA neutralizing antibody (an anti-IL-31RA antibody comprising the H chain of SEQ ID NO: 9 and the L chain of SEQ ID NO: 10);
1.3 mg (1.25 mg to 1.34 mg) of tris(hydroxymethyl)aminomethane;
15 mg (14.5 mg to 15.4 mg) of arginine;
47 mg (46.5 mg to 47.4 mg) of sucrose; and
0.3 mg (0.25 mg to 0.34 mg) of Poloxamer 188.

In one embodiment, regarding the lyophilized formulation of the present disclosure, when the formulation is reconstituted in water, the reconstituted solution comprises:
90 mg/mL to 110 mg/mL, for example 100 mg/mL anti-IL-31RA neutralizing antibody (an anti-IL-31RA antibody comprising the H chain of SEQ ID NO: 9 and the L chain of SEQ ID NO: 10);
16 mmol/L to 24 mmol/L, for example 20 mmol/L Tris-HCl;
120 mmol/L to 180 mmol/L, for example 150 mmol/L Arg-HCl;
225 mmol/L to 275 mmol/L, for example 250 mmol/L sucrose or trehalose; and
0.25 mg/mL to 0.75 mg/mL, for example 0.50 mg/mL Poloxamer 188 or Polysorbate 20,
   where pH is 6 to 8, for example 6.7 to 7.3, and for example 7.

In another embodiment, regarding the lyophilized formulation of the present disclosure, when the formulation is reconstituted in water, the reconstituted solution comprises:
55 mg/mL to 68 mg/mL, for example 61.5 mg/mL anti-IL-31RA neutralizing antibody (an anti-IL-31RA antibody comprising the H chain of SEQ ID NO: 9 and the L chain of SEQ ID NO: 10);
10 mmol/L to 15 mmol/L, for example 12.3 mmol/L Tris-HCl;
74 mmol/L to 110 mmol/L, for example 92 mmol/L Arg-HCl;
139 mmol/L to 169 mmol/L, for example 154 mmol/L sucrose or trehalose; and
0.16 mg/mL to 0.47 mg/mL, for example 0.31 mg/mL Poloxamer 188 or Polysorbate20,
   where pH is 6 to 8, for example 6.7 to 7.3, and for example 7.

Furthermore, in another embodiment, regarding the lyophilized formulation of the present disclosure, when the formulation is reconstituted in water, the reconstituted solution comprises:
45 mg/mL to 55 mg/mL, for example 50 mg/mL anti-IL-31RA neutralizing antibody (an anti-IL-31RA antibody comprising the H chain of SEQ ID NO: 9 and the L chain of SEQ ID NO: 10);
16 mmol/L to 24 mmol/L, for example 20 mmol/L Tris-HCl;
120 mmol/L to 180 mmol/L, for example 150 mmol/L Arg-HCl;
225 mmol/L to 275 mmol/L, for example 250 mmol/L sucrose or trehalose; and
0.25 mg/mL to 0.75 mg/mL, for example 0.50 mg/mL Poloxamer 188 or Polysorbate 20,
   where pH is 6 to 8, for example 6.7 to 7.3, and for example 7.

In still another embodiment, regarding the lyophilized formulation of the present disclosure, when the formulation is reconstituted in water, the reconstituted solution comprises:
23 mg/mL to 28 mg/mL, for example 25 mg/mL anti-IL-31RA neutralizing antibody (anti-IL-31RA antibody comprising the H chain of SEQ ID NO: 9 and the L chain of SEQ ID NO: 10);
16 mmol/L to 24 mmol/L, for example 20 mmol/L Tris-HCl;
120 mmol/L to 180 mmol/L, for example 150 mmol/L Arg-HCl;
225 mmol/L to 275 mmol/L, for example 250 mmol/L sucrose or trehalose; and
0.25 mg/mL to 0.75 mg/mL, for example 0.50 mg/mL Poloxamer 188 or Polysorbate 20,
   where pH is 6 to 8, for example 6.7 to 7.3, and for example 7.

Exemplary embodiments of the formulation of the present disclosure include, but are not limited to, the following formulations:

### Example of the Formulation of the Present Disclosure (The Weight of Each Component

| | Formulation per container | | | Formulation per container | | | Formulation per container | | |
|---|---|---|---|---|---|---|---|---|---|
| | such as syringe | | | such as cartridge | | | such as syringe | | |
| | Target | Minimum | Maximum | Target | Minimum | Maximum | Target | Minimum | Maximum |
| Anti-IL-31RA | 39 mg | 35 mg | 43 mg | 36 mg | 32 mg | 40 mg | 75 mg | 68 mg | 83 mg |
| neutralizing antibody | | | | | | | | | |
| Tris(hydroxymethyl) | 0.9 mg | 0.7 mg | 1.1 mg | 0.9 mg | 0.7 mg | 1.1 mg | 1.8 mg | 1.4 mg | 2.2 mg |
| aminomethane | | | | | | | | | |
| Arginine | 10 mg | 8 mg | 12 mg | 9 mg | 7 mg | 11 mg | 19 mg | 15 mg | 23 mg |
| Sucrose | 33 mg | 30 mg | 36 mg | 31 mg | 28 mg | 34 mg | 64 mg | 58 mg | 70 mg |
| Poloxamer 188 | 0.2 mg | 0.1 mg | 0.3 mg | 0.2 mg | 0.1 mg | 0.3 mg | 0.4 mg | 0.2 mg | 0.6 mg |

| | Formulation per container | | | Formulation per container | | | Formulation per container | | | Formulation per container | | |
|---|---|---|---|---|---|---|---|---|---|---|---|---|
| | such as vial | | | such as vial | | | such as vial | | | such as vial | | |
| | | | Max | | | Max- | | | Max- | | | Max- |
| | Target | Minimum | imum | Target | Minimum | imum | Target | Minimum | imum | Target | Minimum | imum |
| Anti-IL-31RA | 30 mg | 27 mg | 33 mg | 19 mg | 17 mg | 21 mg | 14 mg | 13 mg | 15 mg | 51 mg | 46 mg | 56 mg |
| neutralizing | | | | | | | | | | | | |
| antibody | | | | | | | | | | | | |
| Tris | 3.0 mg | 2.4 mg | 3.6 mg | 1.9 mg | 1.5 mg | 2.3 mg | 1.3 mg | 1.0 mg | 1.6 mg | 2.5 mg | 2.0 mg | 3.0 mg |
| (hydroxymethyl) | | | | | | | | | | | | |
| aminomethane | | | | | | | | | | | | |
| Arginine | 32 mg | 26 mg | 38 mg | 20 mg | 16 mg | 24 mg | 15 mg | 12 mg | 18 mg | 27 mg | 22 mg | 32 mg |
| Sucrose | 104 mg | 94 mg | 114 mg | 66 mg | 59 mg | 73 mg | 47 mg | 42 mg | 52 mg | 88 mg | 79 mg | 97 mg |
| Poloxamer 188 | 0.6 mg | 0.3 mg | 0.9 mg | 0.4 mg | 0.2 mg | 0.6 mg | 0.3 mg | 0.2 mg | 0.5 mg | 0.5 mg | 0.3 mg | 0.8 mg |

### Example of the Formulation of the Present Disclosure (The Concentration of Each Component in the Solution Prior to Lyophilization)

| | Formulation of solution prior to lyophilization | | | Formulation of solution prior to lyophilization | | |
|---|---|---|---|---|---|---|
| | Target | Minimum | Maximum | Target | Minimum | Maximum |
| Anti-IL-31RA neutralizing antibody | 50 mg/mL | 45 mg/mL | 55 mg/mL | 68 mg/mL | 61 mg/mL | 75 mg/mL |
| Tris-HCl or tris(hydroxymethyl) | 10 mmol/L | 8 mmol/L | 12 mmol/L | 13.6 mmol/L | 11 mmol/L | 16 mmol/L |
| aminomethane | | | | | | |
| Arg-HCl or arginine | 75 mmol/L | 60 mmol/L | 90 mmol/L | 102 mmol/L | 82 mmol/L | 122 mmol/L |
| Sucrose or trehalose | 125 mmol/L | 113 mmol/L | 138 mmol/L | 170 mmol/L | 153 mmol/L | 187 mmol/L |
| Poloxamer 188 or Polysorbate 20 | 0.25 mg/mL | 0.13 mg/mL | 0.38 mg/mL | 0.34 mg/mL | 0.17 mg/mL | 0.51 mg/mL |

| | Formulation of solution prior to lyophilization | | | Formulation of solution prior to lyophilization | | |
|---|---|---|---|---|---|---|
| | Target | Minimum | Maximum | Target | Minimum | Maximum |
| Anti-IL-31RA neutralizing antibody | 7.5 mg/mL | 7 mg/mL | 8 mg/mL | 15 mg/mL | 14 mg/mL | 17 mg/mL |
| Tris-HCl or | 6 mmol/L | 5 mmol/L | 7 mmol/L | 6 mmol/L | 5 mmol/L | 7 mmol/L |
| tris(hydroxymethyl)aminomethane | | | | | | |
| Arg-HCl or arginine | 45 mmol/L | 36 mmol/L | 54 mmol/L | 45 mmol/L | 36 mmol/L | 54 mmol/L |
| Sucrose or trehalose | 75 mmol/L | 68 mmol/L | 83 mmol/L | 75 mmol/L | 68 mmol/L | 83 mmol/L |
| Poloxamer 188 or Polysorbate 20 | 0.15 mg/mL | 0.08 mg/mL | 0.23 mg/mL | 0.15 mg/mL | 0.08 mg/mL | 0.23 mg/mL |

### Example of the Formulation of the Present Disclosure (The Concentration of Each Component in the Reconstituted Solution)

| | Formulation of reconstituted solution | | | Formulation of reconstituted solution | | |
|---|---|---|---|---|---|---|
| | Target | Minimum | Maximum | Target | Minimum | Maximum |
| Anti-IL-31RA neutralizing antibody | 61.5 mg/mL | 55 mg/mL | 68 mg/mL | 100 mg/mL | 90 mg/mL | 110 mg/mL |
| Tris-HCl or tris(hydroxymethyl) | 12.3 mmol/L | 10 mmol/L | 15 mmol/L | 20 mmol/L | 16 mmol/L | 24 mmol/L |
| aminomethane | | | | | | |
| Arg-HCl or arginine | 92 mmol/L | 74 mmol/L | 110 mmol/L | 150 mmol/L | 120 mmol/L | 180 mmol/L |
| Sucrose or trehalose | 154 mmol/L | 139 mmol/L | 169 mmol/L | 250 mmol/L | 225 mmol/L | 275 mmol/L |
| Poloxamer 188 or Polysorbate 20 | 0.31 mg/mL | 0.16 mg/mL | 0.47 mg/mL | 0.50 mg/mL | 0.25 mg/mL | 0.75 mg/mL |

| | Formulation of reconstituted solution | | | Formulation of reconstituted solution | | |
|---|---|---|---|---|---|---|
| | Target | Minimum | Maximum | Target | Minimum | Maximum |
| Anti-IL-31RA neutralizing antibody | 25 mg/mL | 23 mg/mL | 28 mg/mL | 50 mg/mL | 45 mg/mL | 55 mg/mL |
| Tris-HCl or tris(hydroxymethyl) | 20 mmol/L | 16 mmol/L | 24 mmol/L | 20 mmol/L | 16 mmol/L | 24 mmol/L |
| aminomethane | | | | | | |
| Arg-HCl or arginine | 150 mmol/L | 120 mmol/L | 180 mmol/L | 150 mmol/L | 120 mmol/L | 180 mmol/L |
| Sucrose or trehalose | 250 mmol/L | 225 mmol/L | 275 mmol/L | 250 mmol/L | 225 mmol/L | 275 mmol/L |
| Poloxamer 188 or Polysorbate 20 | 0.50 mg/mL | 0.25 mg/mL | 0.75 mg/mL | 0.50 mg/mL | 0.25 mg/mL | 0.75 mg/mL |

In a non-limiting embodiment, the formulations of the present disclosure can be used for preventing and/or treating IL-31-associated disorders or symptoms accompanying therewith. In one embodiment, IL-31-associated disorders are inflammatory disorders associated with IL-31 signaling. In a particular embodiment, IL-31-associated disorders are atopic dermatitis, pruritus (e.g., dialytic pruritus or pruritus caused by atopic dermatitis), or prurigo nodularis. In another particular embodiment, the treatment of IL-31-associated disorders or symptoms accompanying therewith is the improvement of sleep disturbance induced by pruritus (e.g., increase in the time from falling asleep to awakening and/or a decrease in sleep onset latency (the time from going to bed to falling asleep)). In another embodiment, the treatment of IL-31-associated disorders or symptoms accompanying therewith is the suppression of at least one symptom caused by atopic dermatitis selected from the group consisting of redness, induration, papules, edema, excoriations, and lichenification. In a particular embodiment, atopic dermatitis is moderate or severe atopic dermatitis for which topical therapy (e.g., treatment by topical steroids or topical calcineurin inhibitors) is not sufficiently effective or is intolerable. In one embodiment, the formulations of the present disclosure are formulations for parental administration, for example, for intravenous administration or for subcutaneous administration.

In a non-limiting embodiment, the present disclosure relates to a method for stabilizing an antibody in an antibody-containing formulation, the method comprising preparing an antibody-containing solution comprising arginine and/or a salt thereof, and/or sucrose and/or trehalose, wherein the molar ratio of arginine and/or a salt thereof to antibody in the solution is 220:1 to 1100:1 and the molar ratio of sucrose or trehalose to the antibody is 370:1 to 1840:1. In another non-limiting embodiment, the present disclosure relates to a method for suppressing antibody aggregation (aggregate formation) in an antibody-containing formulation, the method comprising preparing an antibody-containing lyophilized formulation by freeze-drying an antibody-containing solution comprising 45 mmol/L to 150 mmol/L arginine-hydrochloride (Arg-HCl) and 75 mmol/L to 250 mmol/L sucrose or trehalose. In a further non-limiting embodiment, the present disclosure relates to a method for reducing components with charge heterogeneity in an antibody-containing lyophilized formulation, the method comprising preparing an antibody-containing lyophilized formulation by freeze-drying an antibody-containing solution comprising 45 mmol/L to 150 mmol/L arginine-hydrochloride (Arg-HCl) and 75 mmol/L to 250 mmol/L sucrose or trehalose. In one embodiment, an antibody-containing solution before lyophilization further comprises 6 mmol/L to 20 mmol/L tris(hydroxymethyl)aminomethane-hydrochloride and 0.15 mg/mL to 0.50 mg/mL Poloxamer 188.

In a non-limiting embodiment, the present disclosure relates to a method for preventing and/or treating IL-31-associated disorders, comprising administering an IL-31 antagonist to a subject affected with IL-31-associated disorders or at a risk of being affected with IL-31-associated disorders, the method comprising preparing a lyophilized formulation by freeze-drying a solution comprising 6 mg/mL to 100 mg/mL IL-31 antagonist, 45 mmol/L to 150 mmol/L arginine-hydrochloride (Arg-HCl), and 75 mmol/L to 250 mmol/L sucrose or trehalose: preparing a reconstituted solution by reconstituting the lyophilized formulation: and administering the reconstituted solution to the subject.

In a non-limiting embodiment, the present disclosure relates to use of an IL-31 antagonist in the manufacture of a medicament for prevention and/or treatment of IL-31-associated disorders, characterized in that a lyophilized formulation is prepared by freeze-drying a solution comprising 6 mg/mL to 100 mg/mL IL-31 antagonist, 45 mmol/L to 150 mmol/L arginine-hydrochloride (Arg-HCl), and 75 mmol/L to 250 mmol/L sucrose or trehalose.

In a non-limiting embodiment, the present disclosure relates to an IL-31 antagonist for use in the prevention and/or treatment of IL-31-associated disorders, characterized in that a lyophilized formulation resulting from lyophilizing a solution comprising 6 mg/mL to 100 mg/mL IL-31 antagonist, 45 mmol/L to 150 mmol/L arginine-hydrochloride (Arg-HCl), and 75 mmol/L to 250 mmol/L sucrose or trehalose is reconstituted and used.

In one embodiment, the above-mentioned solution before lyophilization may further comprise 6 mmol/L to 20 mmol/L tris(hydroxymethyl)aminomethane-hydrochloride and 0.15 mg/mL to 0.50 mg/mL Poloxamer 188.

In a non-limiting embodiment, the present disclosure relates to formulations for injection or kits comprising (i) a container: (ii) a lyophilized formulation of the present disclosure: and (iii) optionally water for injection for reconstituting the lyophilized formulation. In one embodiment, a container of the formulations for injection or kits of the present disclosure is a glass syringe, a glass cartridge or a glass vial. In a particular embodiment, the container of formulations for injection or kits of the present disclosure is a dual chamber syringe (DCS) or dual chamber cartridge (DCC), and a lyophilized formulation and water for injection are enclosed in separate compartments in the container, i.e., the lyophilized formulation of the present disclosure is filled into either one of two chambers and water for injection is filled into the other chamber. Preferably, water for injection is water, and optionally satisfies the standards of “water for injection” specified in the Pharmacopoeia of Japan.

IL-31 (interleukin-31) is a T-cell cytokine. It is known that IL-31 is involved in pruritus, and in transgenic mice overexpressing IL-31, dermatitis-like symptoms similar to atopic dermatitis occur, and persistent scratching behavior is observed.

The nucleic acid sequence and amino acid sequence of human IL-31 are also known as RefSeq accession number NM_001014336 and RefSeq accession number NP_001014358, respectively.

The receptor for IL-31 is formed of a heterodimer of IL-31 receptor A (IL-31RA) and oncostatin M receptor (OSMR) (Nat Immunol (2004) 5, 752-60). IL-31RA, also referred to as NR10, is known to have a plurality of splicing variants (WO 00/075314). Known splicing variants are NR10.1 (652 amino acids), NR10.2 (252 amino acids), NR10.3 (662 amino acids, also referred to as IL-31RAv4), IL31RAv3 (764 amino acids), and such and examples of preferred IL-31RA include NR10.3 (IL-31RAv4) and IL-31RAv3. The nucleic acid sequence and amino acid sequence of human IL-31RA (IL-31RAv4) are also known as RefSeq accession number NM_001242638 and RefSeq accession number NP_001229567, respectively. The nucleic acid sequence and the amino acid sequence of human IL-31RA (IL-31RAv3) are also known as RefSeq accession number NM_139017 and RefSeq accession number NP_620586, respectively. The nucleic acid sequence and the amino acid sequence of human OSMR are also known as RefSeq accession number NM_003999 and RefSeq accession number NP_003990, respectively.

The IL-31 antagonist in the present disclosure, in one embodiment, refers to a compound that suppresses or blocks IL-31-induced intracellular signaling. This compound can also be expressed as a compound that inhibits IL-31 signaling. Such a compound may be a naturally occurring compound or an artificially synthesized compound. Moreover, such a compound may be a low-molecular-weight compound or a high-molecular-weight compound such as a protein.

It is known that IL-31 that is present extracellularly triggers intracellular signaling via the IL-31 receptor (heterodimer of IL-31RA and OSMR) present on the cell surface (Nat Immunol (2004) 5, 752-760). The extracellular domain of the IL-31 receptor includes an IL-31-binding domain, and binding of IL-31 thereto causes a change in the conformation of the IL-31 receptor. As a result, intracellular signaling is initiated from the intracellular domain of the IL-31 receptor.

In one method, whether a certain compound inhibits IL-31 signaling can be verified by examining whether the compound inhibits binding of IL-31 to the IL-31 receptor. Examples of methods for making such a determination include an assay using ELISA or flow cytometry and an assay using surface plasmon resonance. With ELISA, for example, whether the compound inhibits the binding of IL-31 to the IL-31 receptor can be evaluated by immobilizing the IL-31 receptor (or IL-31RA) protein onto a plate, preparing a system for detecting the amount of IL-31 protein that binds thereto through the use of a secondary antibody such as an enzyme-labeled anti-IL-31 antibody, and determining whether or not the addition of the compound reduces the amount of detected IL-31 protein.

In an alternative method, whether a certain compound inhibits IL-31 signaling can be verified by examining whether the bioactivity induced by the action of IL-31 on cells is inhibited by the compound. The bioactivity is not particularly limited as long as it can be quantitatively or qualitatively determined using any method, and examples of such bioactivities include cell proliferative activity, protein phosphorylation activity, and gene/protein expression-inducing activity. For example, whether the compound inhibits IL-31 signaling can be evaluated by preparing cells that express the IL-31 receptor on the surface, and whose proliferative activity is induced in response to external IL-31 stimulation, and determining whether or not the addition of the compound reduces the IL-31-induced cell proliferative activity. As such cells, naturally occurring cells inherently expressing the IL-31 receptor may be used, or recombinant cells artificially synthesized to express the IL-31 receptor may be used. A suitable example of recombinant cells includes Ba/F3 cells expressing the IL-31 receptor. As a further alternative, the method described in the document of Dillon et al. (Nat Immunol (2004) 5, 752-760) may be used.

In the present disclosure, the degree of inhibition of IL-31 signaling by the IL-31 antagonist may be, but not limited to, at least 10% or more, preferably 20% or more, 30% or more, 40% or more, 50% or more, 60% or more, 70% or more, and 80% or more, and particularly preferably 90% or more, 95% or more, and 98% or more.

In the present disclosure, a preferred embodiment of the compound that inhibits IL-31 signaling includes a protein that inhibits IL-31 signaling. The protein used herein is not particularly limited as long as it has the property of specifically binding to IL-31 or the IL-31 receptor. Examples of preferred proteins include antibodies and antibody-like molecules (Curr Opin Biotechnol (2006) 17, 653-658; Curr Opin Struct Biol (1997) 7, 463-469; and Protein Sci (2006) 15, 14-27). Antibodies include any antibodies such as monoclonal antibodies (e.g., IgG, IgM, IgE, IgA, and IgD), polyclonal antibodies, engineered antibodies (e.g., chimeric antibodies, humanized antibodies, and glycoengineered antibodies (WO 99/54342 and WO 00/61739)), antibody fragments (e.g., Fab, F(ab′)2, Fv, and CDR), multi-specific antibodies (e.g., bispecific antibodies), and conjugated antibodies (e.g., antibodies conjugated with polyethylene glycol (PEG), radioactive isotopes, or drugs). On the other hand, examples of antibody-like molecules include DARPin (WO 2002/020565), Affibody (WO 1995/001937), Avimer (WO 2004/044011), and Adnectin (WO 2002/032925). More preferred is an antibody that inhibits IL-31 signaling. Examples of other preferred proteins that inhibit IL-31 signaling include a protein containing the extracellular domain of IL-31RA and a protein containing each extracellular domain of the IL-31 receptor (heterodimer of IL-31RA and OSMR).

In the present disclosure, preferred embodiments of the antibody that inhibits IL-31 signaling include an antibody that inhibits IL-31 signaling by binding to IL-31 (anti-IL-31 neutralizing antibody) and an antibody that inhibits IL-31 signaling by binding to the IL-31 receptor (anti-IL-31 receptor neutralizing antibody). Anti-IL-31 receptor neutralizing antibodies include an antibody that inhibits IL-31 signaling by binding to IL-31RA (anti-IL-31RA neutralizing antibody), an antibody that inhibits IL-31 signaling by binding to OSMR (anti-OSMR neutralizing antibody), and an antibody that inhibits IL-31 signaling by binding to the heterodimer of IL-31RA and OSMR (anti-IL-31RA/OSMR heterodimer neutralizing antibody). Of these anti-IL-31 receptor neutralizing antibodies, preferred is an anti-IL-31RA neutralizing antibody or anti-IL-31RA/OSMR heterodimer neutralizing antibody, and more preferred is an anti-IL-31RA neutralizing antibody.

Antibodies used in the present invention are not particularly limited so long as they bind to a desired antigen to inhibit IL-31 signaling, and they may be polyclonal or monoclonal antibodies. Monoclonal antibodies are preferred in that homogeneous antibodies can be stably produced.

Amino acids contained in the amino acid sequences of the present invention may be post-translationally modified (for example, the modification of an N-terminal glutamine into a pyroglutamic acid by pyroglutamylation is well-known to those skilled in the art). Naturally, such post-translationally modified amino acids are included in the antibodies used in the present invention.

The antibody that inhibits IL-31 signaling of the present disclosure in a further embodiment or another embodiment preferably comprises an amino acid variant of an H chain constant region sequence of IgG2, where the amino acid variant comprises glutamic acid at position 419 (EU numbering) in the H chain constant region sequence (SEQ ID NO: 12) of wild-type IgG2. This engineered antibody is advantageous in that it exhibits an increased plasma half-life, compared to a reference antibody comprising an H chain constant region sequence of wild-type IgG2 having the same amino acid sequence except for the amino acid variation at position 419. It is thought that such an increased plasma half-life was caused by a decrease in isoelectric point (pI) induced by the amino acid substitution with glutamic acid at position 419 (Example 2 of WO 2016/167263).

Thus, the lyophilized formulation and solution formulation of the present disclosure in one embodiment is advantageous in that it relates to a lyophilized formulation and solution formulation for providing an increased plasma half-life, compared to a (reference) lyophilized formulation and solution formulation comprising a reference antibody comprising an H chain constant region sequence of wild-type IgG2 having the same amino acid sequence except for the amino acid variation at position 419.

In this case, the antibody that inhibits IL-31 signaling of the present disclosure in a preferred embodiment is any of the following anti-IL-31RA neutralizing antibodies:
(1) an anti-IL-31RA antibody comprising an H chain variable region comprising CDR1 as set forth in SEQ ID NO: 1, CDR2 as set forth in SEQ ID NO: 2, and CDR3 as set forth in SEQ ID NO: 3, and an L chain variable region comprising CDR1 as set forth in SEQ ID NO: 4, CDR2 as set forth in SEQ ID NO: 5, and CDR3 as set forth in SEQ ID NO: 6:
(2) an anti-IL-31RA antibody comprising an H chain variable region as set forth in SEQ ID NO: 7 and an L chain variable region as set forth in SEQ ID NO: 8; and
(3) an anti-IL-31RA antibody comprising an H chain as set forth in SEQ ID NO: 9 and an L chain as set forth in SEQ ID NO: 10.

It is understood that isoelectric point (pI), also simply referred to as “pI”, may be either a theoretical isoelectric point or an experimentally measured isoelectric point, where it is not expressly described in the present specification, and unless it is contradictory in the context.

For example, the value of isoelectric point can be measured by isoelectric focusing known to those skilled in the art. The value of theoretical isoelectric point can be calculated using gene and amino acid sequence analysis software (e.g., Genetyx). Alternatively, the value of theoretical isoelectric point can be measured by performing a pharmacokinetic study of the antibody using, for example, the plasma of mice, rats, rabbits, dogs, monkeys, humans, or the like, in combination with a method known to those skilled in the art such as BIACORE, cell proliferation assay, ELISA, EIA (enzyme immunoassay), RIA (radioimmunoassay), or immunofluorescence.

Whether the plasma half-life of the antibody has changed before and after the amino acid variation (modification) may be verified by performing a pharmacokinetic study of the antibody using a method known to those skilled in the art, using, for example, the plasma of mice, rats, rabbits, dogs, monkeys, humans, or the like.

The antibody that inhibits IL-31 signaling of the present disclosure, in a still further embodiment or another embodiment, preferably does not (substantially) exhibit cross-reactivity with IL-31RA from any of mouse, rat, and rabbit, although it has cross-reactivity with IL-31RA from humans and cynomolgus monkeys.

Methods for preparing antibodies are well known to those skilled in the art, and antibodies can be prepared using the hybridoma method (Nature (1975) 256, 495) or the phage antibody library method (Nature (1991) 352, 624-628: J Mol Biol (1991) 222, 581-597), for example. Using the IL-31 protein or IL-31 receptor protein as an immunogen, a large number of anti-IL-31 antibodies or anti-IL-31 receptor antibodies can be obtained by these methods. Furthermore, screening of these antibodies using any of the above-described methods for detecting the compound that inhibits IL-31 signaling allows an anti-IL-31 neutralizing antibody or an anti-IL-31 receptor neutralizing antibody to be obtained. A protein such as IL-31 or the IL-31 receptor may also be prepared using a genetic engineering technology known to those skilled in the art. Specifically, such a protein can be prepared by inserting a gene encoding a desired protein into an expression vector, introducing the vector into an appropriate host cell, and then purifying the target protein expressed in the host cell or in the culture supernatant of the host cell.

Examples of preferred anti-IL-31 neutralizing antibodies include the anti-IL-31 antibodies described in WO 2006/122079, WO 2008/028192, and WO 2009/071696.

Examples of preferred anti-IL-31RA neutralizing antibodies include, but are not limited to, the anti-IL-31RA (NR10) antibody described in WO 2007/142325, the anti-IL-31RA (NR10) antibody described in WO 2009/072604, and the anti-IL-31RA (NR10) antibody described in WO 2010/064697.

Moreover, examples of other preferred anti-IL-31RA neutralizing antibodies include anti-human IL-31RA (neutralizing) antibodies, specifically including an anti-IL-31RA (neutralizing) antibody that recognizes domain 1 and/or domain 2 of human IL-31RA. As used herein, domain 1 of human IL-31RA designates the region from amino acid at position 53 to amino acid at position 152 (LPAKP to LENIA) in the amino acid sequence as set forth in SEQ ID NO: 11. Domain 2 designates the region from amino acid at position 153 to amino acid at position 259 (KTEPP to EEEAP) in the amino acid sequence as set forth in SEQ ID NO: 11.

Without any limitation, of the anti-IL-31RA neutralizing antibodies, more preferred is the anti-IL-31RA antibody described in WO 2010/064697 comprising an H chain (heavy chain) variable region comprising CDR1 as set forth in SEQ ID NO: 1, CDR2 as set forth in SEQ ID NO: 2, and CDR3 as set forth in SEQ ID NO: 3, and an L chain variable region comprising CDR1 as set forth in SEQ ID NO: 4, CDR2 as set forth in SEQ ID NO: 5, and CDR3 as set forth in SEQ ID NO: 6. Still more preferred is an anti-IL-31RA antibody comprising an H chain variable region as set forth in SEQ ID NO: 7 and an L chain (light chain) variable region as set forth in SEQ ID NO: 8. Particularly preferred is Nemolizumab (CIM331) which is an anti-IL-31RA antibody comprising an H chain as set forth in SEQ ID NO: 9 and an L chain as set forth in SEQ ID NO: 10.

Known methods for defining CDRs include the method according to Kabat et al. (Sequences of Proteins of Immunological Interest, 5th Ed (1991), Bethesda, MD), the method according to Chothia et al. (Science (1986) 233, 755-758), and the method based on antigen-antibody contact regions (J Mol Biol (1996) 262, 732-745). Specifically, each of the methods defines CDRs as follows:

| CDR | Kabat | Chothia | Contact |
|---|---|---|---|
| L1 | L24-L34 | L24-L34 | L30-L36 |
| L2 | L50-L56 | L50-L56 | L46-L55 |
| L3 | L89-L97 | L89-L97 | L89-L96 |
| H1 | H31-H35B | H26-H32/34 | H30-H35B (Kabat numbering) |
| H1 | H31-H35 | H26-H32 | H30-H35 (Chothia numbering) |
| H2 | H50-H65 | H52-H56 | H47-H58 |
| H3 | H95-H102 | H95-H102 | H93-H101 |

An example of a preferred anti-IL-31RA neutralizing antibody of the present disclosure includes an anti-IL-31RA antibody comprising CDR1, CDR2, and CDR3 contained in the H chain variable region as set forth in SEQ ID NO: 7, and CDR1, CDR2, and CDR3 contained in the L chain variable region as set forth in SEQ ID NO: 8, as H chain CDR1 , CDR2, and CDR3, and L chain CDR1, CDR2, and CDR3, respectively. The CDRs in such an antibody may be defined in accordance with any of the method according to Kabat et al., the method according to Chothia et al., and the method based on antigen-antibody contact regions, or in accordance with a combination of these methods.

Similarly, preferred as the anti-IL-31RA neutralizing antibody is an anti-IL-31RA antibody that binds to the same epitope as that of the anti-IL-31RA antibody defined by the above-described sequences of CDRs of the H chain and L chain, H chain and L chain variable region sequences, and full-length H chain and L chain sequences. An epitope refers to a specific structural unit of an antigen to which an antibody recognizes and binds. When the antigen is a polypeptide, the epitope typically consists of about 6 to 10 amino acids. Epitope identification can be performed using a method known to those skilled in the art, for example, a method of synthesizing peptides by fragmentation of the antigen, a method of introducing site-directed mutagenesis into the antigen (e.g., arginine/glutamic acid scanning, J Biol Chem (1995) 270, 21619-21625; J Biol Chem (2006) 281, 20464-20473), and a method of crystallizing an antigen-antibody complex (Using Antibodies: A Laboratory Manual (1999), Cold Spring Harbor Laboratory Press, New York). In the present disclosure, the recitation “binds to the same epitope” means that the epitopes to which two antibodies bind at least partially overlap each other. The degree of the overlap is, but not limited to, at least 10% or more, preferably 20% or more, 30% or more, 40% or more, 50% or more, 60% or more, 70% or more, and 80% or more, particularly preferably 90% or more, and most preferably 100%.

Similarly, preferred as the anti-IL-31RA neutralizing antibody is an anti-IL-31RA antibody that competes for binding to IL-31RA with the anti-IL-31RA antibody defined by the above-described sequences of CDRs of the H chain and L chain, H chain and L chain variable region sequences, and full-length H chain and L chain sequences. Whether the two antibodies compete with each other can be evaluated by using a competition binding assay utilizing ELISA, for example. A specific method is as follows: One of the two antibodies is pre-labeled with, for example, fluorescence. A system for detecting the binding of the antibody (labeled antibody) to the antigen is prepared. A comparison is made between the case where the other unlabeled antibody (test antibody) coexists and the case where the test antibody does not coexist in the system. If the level of binding of the labeled antibody to the antigen is decreased in the presence of the test antibody, it can be judged that the test antibody and the labeled antibody compete with each other. In the present disclosure, the degree of competition is, but not particularly limited to, at least 10% or more, preferably 20% or more, 30% or more, 40% or more, 50% or more, 60% or more, 70% or more, and 80% or more, and particularly preferably 90% or more, 95% or more, and 98% or more (that is, the level of binding of the other antibody is decreased).

Atopic dermatitis in the present disclosure may preferably be atopic dermatitis caused by IL-31 signaling or induced by IL-31, or atopic dermatitis showing responsiveness to the prevention and/or treatment with the IL-31 antagonist, but not limited thereto.

Pruritus in the present disclosure may be atopic dermatitis-induced pruritus, and may preferably be pruritus due to atopic dermatitis caused by IL-31 signaling or induced by IL-31, but not limited thereto. Moreover, pruritus may be pruritus due to atopic dermatitis which shows responsiveness to the prevention and/or treatment with the IL-31 antagonist.

Atopic dermatitis may be moderate to severe atopic dermatitis, for example, and may preferably be moderate or severe atopic dermatitis for which topical therapy is not sufficiently effective or is intolerable, or standard topical therapy is not sufficiently effective or is intolerable, or standard topical therapy is prohibited (for reasons such as contraindications). More preferably, atopic dermatitis may be moderate or severe atopic dermatitis for which topical therapy is not sufficiently effective or is intolerable.

For topical therapy, topical steroids (e.g., glucocorticoids or their derivatives such as prednisolone and hydrocortisone) and topical calcineurin inhibitors known as immunosuppressants (e.g., tacrolimus and pimecrolimus) are known, for example.

In addition to the topical steroids and topical calcineurin inhibitors, cyclosporin, methotrexate (MTX), or azathioprine (AZA), or antihistamines (various drugs are known as antihistaminic preparations, and are broadly classified into first-generation antihistamines and second-generation antihistamines) are known, for example, as therapeutic agents for atopic dermatitis.

More specifically, without any limitation, the following therapeutic methods are known for the treatment of atopic dermatitis (“Therapeutic Guidelines for Atopic Dermatitis”, Furue et al., the Japanese journal of dermatology: 119 (8), pp. 1515-1534, 2009: “Guidelines of care for the management of atopic dermatitis: section 3. Management and treatment with phototherapy and systemic agents.”, Sidbury R et al., J Am Acad Dermatol. (2014), pp. 327-337): and Saeki H, et al., J Dermatol 2009, 36, pp. 563-77).

### (1) Cyclosporin Preparation (Brand Name: Neoral)

Typically, the cyclosporin preparation is orally administered to an adult at 3 mg/kg per day, calculated as cyclosporin, in two divided doses a day. The dose should not exceed 5 mg/kg per day, although it will vary as appropriate depending on the symptoms.

### (2) Steroid Preparation for Oral Administration (Brand Name: Prednisolone Tablets)

Typically, the steroid preparation is orally administered to an adult at 5 mg to 60 mg per day, calculated as prednisolone (in the case of tablets, 1 to 12 tablets; in the case of powder, 0.5 g to 6 g), in one to four divided doses. The dose will vary as appropriate depending on the age or symptoms.

### (3) Ultraviolet Therapy

It is generally said that a patient needs to visit the hospital once or twice a week, although there is no established manual or guidelines.

### (4) Antihistamine Preparation (Brand Name: Allegra)

Typically, the antihistamine preparation is orally administered to an adult at a single dose of 60 mg, calculated as fexofenadine hydrochloride, twice a day. Typically, the antihistamine preparation is orally administered to a child 7 years or older and younger than 12 years at a single dose of 30 mg, calculated as fexofenadine hydrochloride, twice a day, and is orally administered to a child 12 years or older at a single dose of 60 mg, calculated as fexofenadine hydrochloride, twice a day. The dose will vary as appropriate depending on the symptoms.

### (5) Topical Steroid Preparation (Brand Name: Fulmeta)

Typically, an appropriate amount of the topical steroid preparation is applied to an affected area once to several times a day. The amount will vary as appropriate depending on the symptoms.

### (6) Topical Steroid Preparation (Brand Name: Locoid)

Typically, an appropriate amount of the topical steroid preparation is applied once to several times a day. The amount will vary as appropriate depending on the symptoms.

### (7) Tacrolimus Preparation (Brand Name: Protopic)

Typically, an appropriate amount of the tacrolimus preparation is applied to an affected area once or twice a day for an adult. The applied amount should be up to 5 g per application.

### (8) Pimecrolimus Preparation (Brand Name: Elidel)

Typically, an appropriate amount of the pimecrolimus preparation is applied twice a day. The amount will vary as appropriate depending on the symptoms.

The severity (e.g., mild, moderate, or severe) of atopic dermatitis may be classified based on a classification method known to those skilled in the art for scoring the degree of rash or itchiness felt by the subject, such as Shiratori's severity criteria, the below-described Visual Analogue Scale (VAS), Verbal Rating Scale (VRS) for pruritus, SCORing Atopic Dermatitis (SCORAD) established by the European Task Force on Atopic Dermatitis, Eczema Area and Severity Index (EASI) established in the United States, or static Investigator's Global Assessment (sIGA), for example.

The VAS, for example, consists of a 100-mm straight line, on which the subject (patient) indicates the intensity of itchiness at the time of the measurement by drawing a line between 0 to 100 mm, where 0 mm represents no itchiness, and 100 mm represents the worst imaginable itchiness. For example, a subject determined to have a VAS score of 40 mm or more may be recognized as having moderate to severe atopic dermatitis, and in one embodiment, the VAS score may be 45 mm or more, or 50 mm or more. Likewise, in the case of the VRS, for example, a subject classified into the level of “moderate itchiness” or higher may be recognized as having moderate to severe atopic dermatitis (Reich et al. 2012). Alternatively, for example, a subject determined to have an EASI score of 10 or more, a sIGA score of 3 or more, or a total score of 4 or more in the evaluation of the degree of itchiness in the daytime or nighttime based on Shiratori's severity criteria may be recognized as having moderate to severe atopic dermatitis. Alternatively, a subject in which rash with intense inflammation affects, for example, 5% or more of the body surface area may be recognized as having moderate to severe atopic dermatitis. Alternatively, a subject in which one or a combination of a plurality of indices of those mentioned herein are satisfied, as appropriate, may be recognized as having moderate to severe atopic dermatitis.

As used herein, the “subject” may preferably be an animal, and more preferably a mammal (a mouse, a rat, a rabbit, a dog, a monkey (e.g., a cynomolgus monkey), or the like), and particularly preferably a human, but not limited thereto. The human may be an adult (18 years or older) or a child (0 to younger than 18 years, for example, 6 months to younger than 18 years).

In one embodiment, the present disclosure relates to a lyophilized formulation and solution formulation for prevention and/or treatment of atopic dermatitis, comprising an IL-31 antagonist as an active ingredient.

In this case, the IL-31 antagonist may be intended to be repeatedly administered in equal amounts at the same dosing interval, using the predetermined dosing interval and the predetermined dose (dosage) that will be described in detail below.

In one embodiment, the lyophilized formulation and solution formulation of the present disclosure may be used for prevention and/or treatment of pruritus due to atopic dermatitis.

In a still further embodiment or another embodiment, the lyophilized formulation and solution formulation of the present disclosure may be used for improvement of sleep disturbance caused by atopic dermatitis, wherein the sleep disturbance may be caused by pruritus due to atopic dermatitis. The improvement of sleep disturbance may be characterized by, for example, an increase in the time from falling asleep to awakening, and/or a decrease in sleep onset latency (the time from going to bed to falling asleep).

In a still further embodiment or another embodiment, the lyophilized formulation and solution formulation of the present disclosure may be used for suppressing at least one symptom caused by atopic dermatitis selected from the group consisting of redness, induration, papules, edema, excoriations, and lichenification.

In one embodiment of the present disclosure, the prevention and/or treatment of atopic dermatitis may refer to, but not limited to, for example, administering a drug or the like to a subject who currently exhibits atopic dermatitis or various symptoms caused by atopic dermatitis (e.g., pruritus, redness, induration, papules, edema, excoriations, lichenification, decrease in QOL, and lack of sleep) to suppress one or more of these symptoms, and/or, for example, administering a drug or the like to a subject who has previously developed atopic dermatitis or various symptoms caused by atopic dermatitis to eliminate the development or reduce the incidence rate of one or more of these symptoms. The prevention and/or treatment of atopic dermatitis may be judged or determined to be useful for the prevention and/or treatment, as long as it improves any one of the various symptoms caused by atopic dermatitis, even though it cannot prevent and/or treat atopic dermatitis per se.

The subject potentially with atopic dermatitis may be a subject who has had atopic dermatitis in the past, and may have a risk of recurrence of the symptoms, or may be a subject with suspected atopic dermatitis before a doctor or the like makes a diagnosis or determination that the subject has atopic dermatitis, but not limited thereto.

In one embodiment, in some cases, the prevention and treatment of atopic dermatitis may be interpreted synonymously.

It is known that an IL-31 antagonist (CIM331) used in the present Examples demonstrated an improvement in sleep efficiency in the IL-31 antagonist-administered group in a single subcutaneous dose study of the IL-31 antagonist for patients with atopic dermatitis (WO 2016/167263).

Although atopic dermatitis is not necessarily a life-threatening serious disease, the symptoms associated with the disease significantly affect daily life. In particular, pruritus, which is the most characteristic symptom, is an unpleasant sensation that markedly lowers the patient's quality of life (QOL), and has been reported to hinder the patient's sleep (Zuberbier T, Orlow S J, Paller A S, Taieb A, Allen R, Hernanz-Hermosa J M, Ocampo-Candiani J, Cox M, Langeraar J, Simon J C. Patient perspective on the management of atopic dermatitis. J Allergy Clin Immunol 2006; 118: 226-32.). Furthermore, when the patient is a child, there is a significant burden not only on the affected child but also on the parents, and there is a report that the parents of a child with moderate or severe atopic dermatitis spend 3 hours every day in therapeutic treatment, and lose 1 to 2 hours of sleep every day (Su J C, Kemp A S, Varigos G A, Nolan T M. Atopic eczema: its impact on the family and financial cost. Arch Dis Chil 1997; 76: 159-62.).

In another embodiment, therefore, the present disclosure relates to a lyophilized formulation and solution formulation for prevention and/or treatment of atopic dermatitis comprising an IL-31 antagonist as an active ingredient, which is further for improvement of sleep disturbance caused by atopic dermatitis. Alternatively, in a further embodiment or another embodiment, the present disclosure relates to a lyophilized formulation and solution formulation for improvement of a decrease in QOL caused by atopic dermatitis. The improvement of sleep disturbance may be characterized by, for example, an increase in the time from falling asleep to awakening, and/or a decrease in sleep onset latency (the time from going to bed to falling asleep).

As used herein, the recitation “repeatedly administered in equal amounts at the same dosing interval” intends to mean that the dose at which the IL-31 antagonist of the present disclosure is initially administered to a subject (initial dose) is equal to a continuous dose at which the IL-31 antagonist is subsequently administered (namely, the dose to be continuously administered subsequent to the administration of the initial dose), and the IL-31 antagonist is administered at an equal dosing interval (interval between doses). Specifically, for example, the above-described recitation means that the interval between the administration of the initial dose and the administration of the first continuous dose, or every interval between the administration of the n-th (n is an integer of 1 or more) continuous dose and the administration of the (n+1)-th continuous dose is equal, and the doses are equal. Those skilled in the art will naturally understand that, for decided dosing intervals (e.g., every 4 weeks in the case where the dosing interval is decided to be every 4 weeks), each dosing interval has a “tolerable range”, and those skilled in the art can decide the tolerable range, as appropriate.

In the present invention, the term “stable antibody-containing formulation” refers to a formulation in which aggregates and/or components with charge heterogeneity from proteins such as antibodies are difficult to be generated, i.e., the formulations in which deterioration reactions, including generation of insoluble aggregates, soluble aggregates, components with charge heterogeneity, are difficult to occur in the formulation.

“Components with charge heterogeneity” refer to components having protein surface charges that are different from those of the major component due to deamidation, oxidation, hydrolysis, and such.

Amounts of aggregates can be measured by size exclusion chromatography (SEC), SDS polyacrylamide gel electrophoresis (SDS-PAGE), capillary SDS gel electrophoresis (CE-SDS), dynamic light scattering (DLS), light obscuration automated microparticle counter (HIAC), flow imaging, Analytical Ultracentrifuge (AUC) and such, and measurements by size exclusion chromatography (SEC) are preferred in the present invention. It is thought that as measurement conditions, samples are measured using a column (TOSOH, TSKgel G3000SWXL), using 50 mmol/L phosphate buffer (pH 7.0), 300 mmol/L sodium chloride, 0.05% sodium azide as a mobile phase at a flow rate of 0.5 mL/min, but conditions are not limited thereto. In one embodiment, amounts of aggregates are measured by the methods described in Examples herein.

Components with charge heterogeneity can be measured by ion exchange chromatography (IEC), specifically cation exchange chromatography or anion exchange chromatography, isoelectric focusing and such, and in the present invention, the measurements are preferably measurements by anion exchange chromatography. Amounts of components with charge heterogeneity in samples can be measured by ion exchange chromatography (IEC) using a column (TOSOH, TSKgel DEAE-NPR), using 25 mmol/L Tris-HCl buffer (pH 7.5) as mobile phase A and 25 mmol/L Tris-HCl buffer (pH 7.5), 250 mmol/L sodium chloride as mobile phase B at a flow rate of 1.0 mL/min, but methods are not limited thereto. In one embodiment, components with charge heterogeneity are measured by the methods described in Examples herein.

In the present invention, “polypeptide” generally refers to peptides and proteins having a length of approximately ten amino acids or longer. Ordinarily, they are biologically derived polypeptides, but are not particularly limited thereto, and may be, for example, polypeptides comprising an artificially designed sequence. Furthermore, they may be any naturally-occurring polypeptides, synthetic polypeptides, recombinant polypeptides, or such. Additionally, fragments of the above-mentioned polypeptides are also included in the polypeptides of the present invention.

The term “antibody” is used in the broadest sense, and includes monoclonal antibodies, polyclonal antibodies, dimers, multimers, multispecific antibodies (such as bispecific antibodies), antibody derivatives, and modified antibodies (Miller K et al. J Immunol. 2003, 170(9), 4854-61) so long as they show a desired biological activity. The antibodies may be mouse antibodies, human antibodies, humanized antibodies, chimeric antibodies, or those derived from another species, or artificially synthesized antibodies. The antibodies disclosed herein can be of any type (for example, IgG, IgE, IgM, IgD, and IgA), class (for example, IgG1, IgG2, IgG3, IgG4, IgA1 and IgA2) or subclass of immunoglobulin molecules. The immunoglobulins can be derived from any species (for example, human, mouse, or rabbit). The terms “antibody”, “immune globulin” and “immunoglobulin” are used interchangeably in a broad sense.

Recombinant antibodies produced by using genetic engineering techniques can be used as the antibodies. A recombinant antibody can be obtained by cloning a DNA encoding the antibody from hybridomas or antibody-producing cells such as sensitized lymphocytes that produce antibodies: inserting this into a vector; and then introducing it into hosts (host cells) to produce the antibody.

Antibodies of the present invention can be produced by methods known to those skilled in the art. Specifically, a DNA encoding the antibody of interest is inserted into an expression vector. The insertion into the expression vector is carried out such that the expression will take place under the control of expression regulatory regions such as an enhancer and a promoter. Next, host cells are transformed using this expression vector to express the antibody. Appropriate combinations of a host and an expression vector can be used in this case.

The antibodies of the present invention thus obtained can be isolated from the inside of host cells or the outside of the cells (medium, etc.), and purified to be substantially pure, homogeneous antibodies. The antibodies can be separated and purified by methods ordinarily used for separating and purifying antibodies, and the methods are not limited in any way. For example, the antibodies can be separated and purified by appropriately selecting and combining column chromatography, filtration, ultrafiltration, salting-out, solvent precipitation, solvent extraction, distillation, immunoprecipitation, SDS-polyacrylamide gel electrophoresis, isoelectrofocusing, dialysis, recrystallization, and such.

The present inventors examined the effects of various additives to evaluate stability of samples containing the above-mentioned anti-IL-31RA antibody (CIM331) during storage by thermal acceleration test. As a result, the present inventors found that by preparing lyophilized formulations from solutions added with arginine-hydrochloride and sucrose or trehalose, aggregate formation was suppressed compared to when they do not comprise stabilizers or bulking agents (fillers), when sodium chloride is added as a stabilizer, or when other sugars are added as a bulking agent. The present inventors also found that subvisible particles formation and aggregate formation are suppressed by adding Poloxamer 188 and/or Polysorbate 20, which are nonionic surfactants, to an antibody-containing solution. Furthermore, the present inventors found that aggregate formation and components with charge heterogeneity are suppressed at pH 6 to pH 8 in antibody-containing solutions comprising arginine-hydrochloride, sucrose or trehalose, and nonionic surfactant.

The concentration (amount) of arginine in formulations of the present invention is preferably 45 mM to 150 mM. Examples of the arginine concentration (amount) include 45 mM, 75 mM, 92 mM, 102 mM and 150 mM.

The solution pH of a formulation of the present invention is preferably 6 to 8, more preferably 6.5 to 7.5, and even more preferably 7.

The formulations of the present invention can comprise as a buffer, for example, Tris buffer. Tris buffer includes, for example, tris(hydroxymethyl)aminomethane and/or salts thereof, e.g., tris(hydroxymethyl)aminomethane-hydrochloride, tris(hydroxy methyl)aminomethane-aspartate, tris(hydroxymethyl)aminomethane-glutamate or tris(hydroxy methyl)aminomethane-acetate. Amount of Tris buffer added to the formulation of the present invention is preferably 6 mM to 20 mM, and for example, 6 mM, 10 mM, 12.3 mM, 13.6 mM, or 20 mM.

Surfactants contained in formulations of the present invention are, for example Polysorbate 20 (PS20), and Pluronic F-68 (Poloxamer 188: polyoxyethylene (160) polyoxypropylene (30) glycol), and Poloxamer 188 is particularly preferred. The amount of Poloxamer 188 (or PX188) added to a formulation of the present invention is preferably 0.15 mg/mL to 0.5 mg/mL. Examples of the amount of Poloxamer 188 added to the formulation include 0.15 mg/mL, 0.25 mg/mL, 0.31 mg/mL, 0.34 mg/mL, and 0.50 mg/mL.

The formulations of the present invention may further contain sugars. Preferred sugars used in the present invention are sucrose, trehalose, mannitol, and lactose, and sucrose and trehalose are particularly preferred.

The amount of sugars added to the formulations of the present invention is generally 1 mM to 1000 mM, preferably 5 mM to 500 mM, and more preferably 10 mM to 300 mM, for example 75 mM to 250 mM, and for example 75 mM, 125 mM, 154 mM, 170 mM, or 250 mM.

If needed, the formulations of the present invention may additionally contain appropriate cryoprotectants, suspending agents, solubilizing agents, tonicity agents, preservatives, adsorption inhibitors, diluents, excipients, pH adjustors, analgesics, sulfur-containing reducing agents, antioxidants, and such.

Cryoprotectants include, for example, sugars such as trehalose, sucrose, and sorbitol.

Solubilizing agents include, for example, polyoxyethylene hardened castor oil, Polysorbate 80, nicotinamide, polyoxyethylene sorbitan monolaurate, macrogol, and castor oil fatty acid ethyl ester.

Tonicity agents include, for example, sodium chloride, potassium chloride, and calcium chloride.

Preservatives include, for example, methyl-p-hydroxy benzoate, ethyl-p-hydroxy benzoate, sorbic acid, phenol, cresol, and chlorocresol.

Adsorption inhibitors include, for example, human serum albumin, lecithin, dextran, ethylene oxide/propylene oxide copolymer, hydroxypropyl cellulose, methyl cellulose, polyoxyethylene hardened castor oil, and polyethylene glycol.

Sulfur-containing reducing agents include, for example, those containing sulfhydryl groups such as N-acetylcysteine, N-acetylhomocysteine, thioctic acid, thiodiglycol, thioethanol amine, thioglycerol, thiosorbitol, thioglycolic acid and salts thereof, sodium thiosulfate, glutathione, and thioalkanoic acids having one to seven carbon atoms.

Antioxidants include, for example, erythorbic acid, dibutylhydroxytoluene, butylhydroxyanisole, α-tocopherol, tocopherol acetate, L-ascorbic acid and salts thereof, L-ascorbic acid palmitate, L-ascorbic acid stearate, sodium hydrogen sulfite, sodium sulfite, triamyl gallate, propyl gallate, and chelating agents such as disodium ethylenediamine tetraacetate (EDTA), sodium pyrophosphate, and sodium metaphosphate.

The antibody-containing formulations of the present invention can be administered to a patient via any appropriate route, for example, by bolus injection or continuous infusion for a certain period, intravenously, intramuscularly, or subcutaneously. Intravenous administration or subcutaneous administration is preferred.

Dose of Nemolizumab (CIM331) is, for example, 0.001 mg/kg to 1000 mg/kg, and dosing interval is at least one day or longer.

More specifically, for example, Nemolizumab (CIM331) is administered repeatedly to a subject affected with atopic dermatitis or at a risk of being affected with atopic dermatitis at 0.1 mg to 1000 mg/body/2 weeks, 0.1 mg to 1000 mg/body/4 weeks, or 0.1 mg to 1000 mg/body/8 weeks, or 0.01 mg to 10 mg/kg/2 weeks, 0.01 mg to 10 mg/kg/4 weeks, or 0.01 mg to 10 mg/kg/8 weeks, preferably 0.5 mg/kg/4 weeks, or 50 mg to 75 mg/body/4 weeks in equal amounts and at the same dosing interval. Alternatively, Nemolizumab (CIM331) is repeatedly administered at 60 mg/body/4 weeks at the same dose and at the same dosing interval. Alternatively, Nemolizumab (CIM331) is administered at a dosing interval of four weeks, and repeatedly administered at 60 mg/body as an initial dose and at 30 mg/body as a continuous dose. Those skilled in the art will naturally understand that when a lyophilized formulation comprising one dose Nemolizumab (CIM331) is enclosed in a container (vial, cartridge or syringe), considering the loss of reconstituted drug solution when administered, an excess amount of lyophilized formulation would be filled to ensure, from one container (vial, cartridge or syringe), the amount sufficient to administer one dose Nemolizumab (CIM331).

Another embodiment of the present invention is a method for stabilizing an antibody in an antibody-containing formulation. For example, the embodiment is a method for stabilizing an antibody in an antibody-containing solution formulation, the method comprising adding arginine and/or salts thereof, and sucrose and/or trehalose to the solution. In addition, for example the embodiment is a method for stabilizing an antibody in an antibody-containing lyophilized formulation, the method comprising preparing an antibody-containing lyophilized formulation by freeze-drying an antibody-containing solution comprising arginine and/or salts thereof, and sucrose and/or trehalose.

In addition, another embodiment of the present invention is a method for suppressing antibody aggregation (aggregate formation) in an antibody-containing formulation. For example, the embodiment is a method for suppressing antibody aggregation (aggregate formation) in an antibody-containing solution formulation, the method comprising adding arginine and/or salts thereof, and sucrose and/or trehalose to the solution. Furthermore, for example, the embodiment is a method for suppressing antibody aggregation (aggregate formation) in an antibody-containing lyophilized formulation, the method comprising preparing an antibody-containing lyophilized formulation by freeze-drying an antibody-containing solution comprising arginine and/or salts thereof, and sucrose and/or trehalose.

Another embodiment of the present invention is a method for reducing components with charge heterogeneity in an antibody-containing formulation. For example, the embodiment is a method for reducing components with charge heterogeneity in an antibody-containing solution formulation, the method comprising adding arginine and/or salts thereof, and sucrose and/or trehalose to the solution. Furthermore, for example, the embodiment is a method for reducing components with charge heterogeneity in an antibody-containing lyophilized formulation, the method comprising preparing an antibody-containing lyophilized formulation by freeze-drying an antibody-containing solution comprising arginine and/or salts thereof, and sucrose and/or trehalose.

Furthermore, in the above-mentioned methods for stabilizing an antibody, methods for suppressing antibody aggregation (aggregates formation), and methods for reducing components with charge heterogeneity, concentration of arginine-hydrochloride (Arg-HCl) in the solution is preferably 45 mmol/L to 150 mmol/L, concentration of sucrose or trehalose in a solution is preferably 75 mmol/L to 250 mmol/L.

In the above-mentioned methods for stabilizing an antibody, methods for suppressing antibody aggregation (aggregate formation), and methods for reducing components with charge heterogeneity, the antibody is preferably Nemolizumab (CIM331).

As used herein, aspects referred to by the expression “comprising” include those referred to by the expression “essentially consisting of”, and those referred to by the expression “consisting of”.

Numerical values recited herein may vary within a certain range, for example, depending on the instruments or equipment, measurement conditions, and procedure used by those skilled in the art, and so long as they are within a range that allows the objective of the invention to be accomplished, they may encompass a deviation of approximately 10%, for example.

All patents and references explicitly cited herein are incorporated by reference into this specification in its entirety.

The present invention will be further illustrated by the Examples below, but it is not to be construed as being limited thereto.

### EXAMPLE

### Definition

### Experimental Methods

To adjust pH, formulations prepared in each Example contain 6 mmol/L to 20 mmol/L Tris-HCl, which do not contribute to the stability of CIM331 (an anti-IL-31RA antibody comprising the H chain of SEQ ID NO: 9 and the L chain of SEQ ID NO: 10).

Thermal acceleration study of solution formulations was performed at 40° C./75% RH. Meanwhile, as lyophilized formulations are generally more stable, thermal acceleration study of lyophilized formulations was performed at a higher temperature, 50° C./75% RH.

In Example 3, to verify effects of nonionic surfactants, shaking study was performed. Analysis Methods

Regarding measurements of amounts of CIM331 aggregate, considering the accuracy of analysis methods, it can be judged that there is significant difference when the percentage of aggregates has 0.07% or higher difference.

Regarding measurements of components with charge heterogeneity of CIM331, considering the accuracy of analysis methods, it can be judged that there is significant difference when the difference is 1.3% or higher for Basic region, 1.4% or higher for Main region, and 0.6% or higher for Acidic region.

Since stability is different between solution formulation and lyophilized formulation, results from analysis methods cannot similarly be interpreted between solution formulation and lyophilized formulation.

### Example 1

### Aggregate Suppressing Effects of Arginine on the Humanized IgG2 Antibody CIM331 During Thermal Accelerated Storage

### [1-1] Stability Assessment of Lyophilized Formulations

### (1) Materials

CIM331 is a monoclonal antibody that binds to IL-31RA, and is a humanized IgG2 antibody expected to lead to the treatment of atopic dermatitis and such, by inhibiting the function of IL-31, which is identified as a pruritus-inducible cytokine.

### (2) Test Samples

Various prepared solutions comprising 30 mg/mL CIM331, 6 mmol/L Tris-HC1, 75 mmol/L Sucrose, 0.15 mg/mL Poloxamer 188, and either one of 45 mmol/L Arg-HCl or 45 mmol/L NaCl as a stabilizer, and a prepared solution not comprising a stabilizer were prepared and filled into glass vials (2 mL/vial). Filled drug solutions were lyophilized under the condition shown below.

| Temperature | Time | | Pressure |
|---|---|---|---|
| Room temperature → −45° C. | 1 | hour | — |
| −45° C. | 6 | hours | — |
| −45° C. → 0° C. | 50 | minutes | 10 Pa |
| 0° C. | 50 | hours | 10 Pa |
| 0° C. → 30° C. | 30 | minutes | 6 Pa |
| 30° C. | 10 | hours | 6 Pa |

After humanized antibody-containing lyophilized formulations prepared as such were left to stand in a 50° C./75% RH incubator for 8 weeks, reconstituted solutions prepared such that the CIM331 concentration became 100 mg/mL by adding ultra-pure water were used as test samples.

### (3) Method for Measuring Amounts of CIM331 Aggregates and Method for Calculating Amounts of CIM331 Aggregates

Samples were measured for their amounts of aggregates by size exclusion chromatography (SEC) at a flow rate of 0.5 mL/min using a column (TOSOH, TSKgel G3000SWXL), and 50 mmol/L phosphate buffer (pH 7.0), 300 mmol/L sodium chloride, 0.05% sodium azide as a mobile phase.

Among detected peaks, a peak showing the maximum area and height is defined as monomer body, and peaks detected before the monomer body were collectively defined as aggregates (HMWS).

For all peaks, area was calculated and peak area ratio of target peak was determined according to the equation below.

$$Peak area ratio of target peak ( \% ) = \frac{Peak area of target peak}{Peak area of target peak + sum of other peak areas} \times 100$$

### (4) Results

The obtained results are shown in Table 1.

TABLE 1

| Stabilizer | | Arg-HCl | Without stabilizer | NaCl |
|---|---|---|---|---|
| HMWS | Initial | 0.52 | 0.61 | 0.59 |
| [%] | 50° C. after 8 weeks | 1.30 | 3.79 | 2.10 |

| | | | |
|---|---|---|---|
| Relative increase rate [%]* | 24.5 | 100.0 | 47.5 |

*: From HMWS increased amount when a stabilizer was not added and HMWS increased amount when each stabilizer was added, relative increase rates compared to when a stabilizer was not added were calculated.

As is apparent from Table 1, a sample added with arginine-hydrochloride showed high aggregate-suppressing effect compared to a sample not added with a stabilizer and a sample added with sodium chloride eight weeks after 50° C./75% RH thermal acceleration.

### [1-2] Stability Assessment of Solution Formulations

### (1) Materials

The antibody described in [1-1] was used.

### (2) Test Samples

Various prepared solutions comprising 100 mg/mL CIM331, 20 mmol/L Tris-HCl, 250 mmol/L Sucrose, 0.5 mg/mL Poloxamer 188, pH 7, and either one of 150 mmol/L Arg-HCl or 150 mmol/L NaCl as a stabilizer, and a prepared solution not comprising a stabilizer were prepared and filled into glass vials (1 mL/vial). After humanized antibody-containing solution formulations prepared as such were left to stand in a 40° C./75% RH incubator for four weeks, they were used as test samples.

### (3) Method for Measuring Amounts of CIM331 Aggregates and Method for Calculating Amounts of CIM331 Aggregates

The methods were performed according to the methods described in [1-1].

### (4) Results

The obtained results are shown in Table 2.

TABLE 2

| | |
|---|---|
| Arg- | Without |

| Stabilizer | | HCl | stabilizer | NaCl |
|---|---|---|---|---|
| HMWS | Initial | 0.55 | 0.63 | 0.65 |
| [%] | 40° C. after 4 weeks | 1.00 | 1.69 | 1.48 |

| | | | |
|---|---|---|---|
| Relative increase rate [%]* | 42.5 | 100.0 | 78.3 |

*: From HMWS increased amount when a stabilizer was not added and HMWS increased amount when each stabilizer was added, relative increase rates compared to when a stabilizer was not added were calculated.

As is apparent from Table 2, samples added with arginine hydrochloride showed high aggregate-suppressing effect compared to a sample not added with a stabilizer and a sample added with sodium chloride four weeks after 40° C./75% RH thermal acceleration.

### Example 2

### Aggregate-Suppressing Effects of Sucrose and Trehalose on the Humanized IgG2 Antibody CIM331 During Thermal Accelerated Storage

### (1) Materials

The antibody described in Example 1 was used.

### (2) Test Samples

Various prepared solutions comprising 30 mg/mL CIM331, 6 mmol/L Tris-HCl, 45 mmol/L Arg-HC1, 0.15 mg/mL Poloxamer 188, and any one of 75 mmol/L Sucrose, 75 mmol/L Mannitol, 75 mmol/L Glucose, 75 mmol/L Lactose or 75 mmol/L Trehalose as a bulking agent, and a prepared solution not comprising a bulking agent were prepared and filled into glass vials (2 mL/vial). Filled drug solutions were lyophilized under the condition shown below.

| Temperature | Time | | Pressure |
|---|---|---|---|
| Room temperature → −45° C. | 1 | hour | — |
| −45° C. | 6 | hours | |
| −45° C. → 0° C. | 50 | minutes | 10 Pa |
| 0° C. | 50 | hours | 10 Pa |
| 0° C. → 30° C. | 30 | minutes | 6 Pa |
| 30° C. | 10 | hours | 6 Pa |

After humanized antibody-containing lyophilized formulations prepared as such were left to stand in a 50° C./75% RH incubator for eight weeks, reconstituted solutions prepared by adding ultra-pure water such that the CIM331 concentration became 100 mg/mL were used as test samples.

### (3) Method for Measuring Amounts of CIM331 Aggregates and Method for Calculating Amounts of CIM331 Aggregates

The methods were performed according to the methods described in Example 1.

### (4) Results

The obtained results are shown in Table 3.

TABLE 3

| | | | Without | | | | |
|---|---|---|---|---|---|---|---|
| | | | bulking | | | | |
| Bulking agent | | Sucrose | agent | Mannitol | Glucose | Lactose | Trehalose |
| HMWS | Initial | 0.52 | 0.71 | 0.50 | 0.53 | 0.54 | 0.50 |
| [%] | 50° C. after | 1.30 | 8.51 | 2.87 | 13.25 | 4.27 | 1.96 |
| | 8 weeks | | | | | | |

| | | | | | | |
|---|---|---|---|---|---|---|
| Relative increase rate [%]* | 10.0 | 100.0 | 30.4 | 163.1 | 47.8 | 18.7 |

*From HMWS increased amount when a bulking agent was not added and HMWS increased amount when each bulking agent was added, relative increase rates compared to when a bulking agent was not added were calculated.

As is apparent from Table 3, samples added with sucrose or trehalose both showed relative increase rate of the 10% range, and showed high aggregate-suppressing effect compared to a sample not added with a bulking agent and samples added with other bulking agents eight weeks after 50° C./75% RH thermal acceleration.

### Example 3

### Subvisible Particles Formation-Suppressing Effects and Solution State-Stabilizing Effects of Nonionic Surfactants on the Humanized IgG2 Antibody CIM331 During Shaking Stress Test

### (1) Materials

The antibody described in Example 1 was used.

### (2) Test Samples

Various prepared solutions comprising 100 mg/mL CIM331, 20 mmol/L Tris-HCl, pH 7, 150 mmol/L Arg-HCl, 250 mmol/L Sucrose, and either one of 0.5 mg/mL Poloxamer 188 or 0.5 mg/mL Polysorbate 20 as a nonionic surfactant, and a prepared solution not comprising a nonionic surfactant were prepared and filled into glass vials (1 mL/vial). After humanized antibody-containing solution formulations prepared as such were shaken at room temperature for three days at a speed of approximately 200 rpm, they were used as test samples.

### (3) Method for Measuring Subvisible Particles

Number of subvisible particles were counted using a liquid particle counter (Hach Ultra Analytics, Model 9703).

### (4) Method for Measuring Amounts of CIM331 Aggregates and Method for Calculating Amounts of CIM331 Aggregates

The methods were performed according to the methods described in Example 1.

### (5) Results

The obtained results are shown in Tables 4 and 5.

TABLE 4

| | | | Without | |
|---|---|---|---|---|
| | | Poloxamer | nonionic | Polysorbate |
| Surfactant | | 188 | surfactant | 20 |
| Subvisible particles | Initial | 3 | 3 | 21 |
| counts | 200 rpm | 3 | 404200 | 11 |
| (≥10 μm) | after 3 days | | | |
| Subvisible particles | Initial | 1 | 1 | 7 |
| counts | 200 rpm | 1 | 26640 | 3 |
| (≥25 μm) | after 3 days | | | |

TABLE 5

| | | | Without | |
|---|---|---|---|---|
| | | Poloxamer | nonionic | Polysorbate |
| Surfactant | | 188 | surfactant | 20 |
| HMWS | Initial | 0.55 | 0.53 | 0.51 |
| [%] | 200 rpm | 0.61 | 1.17 | 0.56 |
| | after 3 days | | | |

| | | | |
|---|---|---|---|
| Relative increase rate [%]* | 9.4 | 100.0 | 7.8 |

*: From HMWS increased amount when a nonionic surfactant was not added and HMWS increased amount when each nonionic surfactant was added, relative increase rates compared to when a nonionic surfactant was not added were calculated.

As is apparent from Tables 4 and 5, samples containing Poloxamer 188 or Polysorbate 20, which is a nonionic surfactant, showed high subvisible particles formation-suppressing effect and high aggregate-suppressing effect in samples after shaking at room temperature for three days at a speed of approximately 200 rpm.

### Example 4

### Effects of pH on Stability of the Humanized IgG2 Antibody CIM331 During Thermal Accelerated Storage

### (1) Materials

The antibody described in Example 1 was used.

### (2) Test Samples

Various prepared solutions comprising 100 mg/mL CIM331, 20 mmol/L Tris-HCl, 150 mmol/L Arg-HC1, 250 mmol/L Sucrose, and 0.5 mg/mL Poloxamer 188, of which pH is any one of pH 6, pH 7 or pH 8, were prepared and filled into glass vials (1 mL/vial). After humanized antibody-containing solution formulations prepared as such were left to stand in a 40° C./75% RH incubator for four weeks, they were used as test samples.

### (3) Method for Measuring Amounts of CIM331 Aggregates and Method for Calculating Amounts of CIM331 Aggregates

The methods were performed according to the methods described in Example 1.

(4) Methods for Measuring and Calculating Components with Charge Heterogeneity of CIM331

Samples were measured for their amounts of components with charge heterogeneity by ion-exchange chromatography (IEC) at a flow rate of 1.0 mL/min using column (TOSOH, TSKgel DEAE-NPR), and 25 mmol/L Tris-HCl buffer (pH 7.5) as mobile phase A and 25 mmol/L Tris-HCl buffer (pH 7.5), 250 mmol/L sodium chloride as mobile phase B.

Among detected peaks, a peak showing maximum area and height was defined as Main region, peaks detected before Main region were collectively defined as Basic region, peaks detected after Main region were collectively defined as Acidic region.

For all peaks, area was calculated, and peak area ratio of target peak was determined according to the equation below.

$$Peak area ratio of target peak ( \% ) = \frac{Peak area of target peak}{Peak area of target peak + sum of other peak areas} \times 100$$

### (5) Results

The obtained results are shown in Table 6.

TABLE 6

| pH | | pH6 | pH7 | pH8 |
|---|---|---|---|---|
| HMWS | Initial | 0.51 | 0.55 | 0.56 |
| [%] | 40° C. after 4 | 1.16 | 1.00 | 2.07 |
| | weeks | | | |
| Main | Initial | 79.9 | 79.8 | 79.7 |
| region [%] | 40° C. after 4 | 32.9 | 49.8 | 43.1 |
| | weeks | | | |

As is apparent from Table 6, samples of pH 6 to pH 8 showed sufficient stability four weeks after 40° C./75% RH thermal acceleration, and in particular in a sample at pH 7, high aggregate-suppressing effects and effects of suppressing components with charge heterogeneity were achieved.

### Example 5

### Effects of Humanized IgG2 Antibody CIM331 Concentration and Concentrations of Other Formulation Components on Stability During Thermal Accelerated Storage

### (1) Materials

The antibody described in Example 1 was used.

### (2) Test Samples

Various prepared solutions comprising 100 mg/mL or 50 mg/mL CIM331, 20 mmol/L or 10 mmol/L Tris-HC1, 150 mmol/L or 75 mmol/L Arg-HCl as a stabilizer, 250 mmol/L or 125 mmol/L Sucrose as a bulking agent, and 0.5 mg/mL or 0.25 mg/mL Poloxamer 188 as a nonionic surfactant, and prepared solutions comprising 6 mmol/L Tris-HC1, 45 mmol/L Arg-HCl, 75 mmol/L Sucrose, 0.15 mg/mL Poloxamer 188, and 30 mg/mL, 15 mg/mL or 6 mg/mL CIM331 were prepared and filled into glass vials (2 mL/vial). Filled drug solutions were lyophilized under the condition shown below.

| Temperature | Time | | Pressure |
|---|---|---|---|
| Room temperature → −45° C. | 1 | hour | — |
| −45° C. | 6 | hours | — |
| −45° C. → 0° C. | 50 | minutes | 10 Pa |
| 0° C. | 50 | hours | 10 Pa |
| 0° C. → 30° C. | 30 | minutes | 6 Pa |
| 30° C. | 10 | hours | 6 Pa |

After humanized antibody-containing lyophilized formulations prepared as such were left to stand in a 50° C./75% RH incubator for eight weeks, reconstituted solutions prepared by adding ultra-pure water such that CIM331 concentrations became as shown below were used as test samples.

| | CIM331 Concentration [mg/mL] | | | | |
|---|---|---|---|---|---|
| Solution before lyophilization | 100 | 50 | 30 | 15 | 6 |
| Reconstituted solution | 100 | 50 | 100 | 50 | 20 |

### (3) Method for Measuring Amounts of CIM331 Aggregates and Method for Calculating Amounts of CIM331 Aggregates

The methods were performed according to the methods described in Example 1.

### (4) Methods for Measuring and Calculating Components With Charge Heterogeneity of CIM331

The methods were performed according to the methods described in Example 4.

### (5) Results

The obtained results are shown in Table 7.

TABLE 7

| CIM331 | | | | | | |
|---|---|---|---|---|---|---|
| Concentration | | | | | | |
| [mg/mL] | | | | | | |
| (Before | | | | | | |
| lyophilization) | | 100 | 50 | 30 | 15 | 6 |
| Formulation | | 20 mmol/L | 10 mmol/L | 6 mmol/L | | |
| components | | Tris-HCl, | Tris-HCl, | Tris-HCl, | | |
| | | 150 mmol/L | 75 mmol/L | 45 mmol/L | | |
| | | Arg-HCl, | Arg-HCl, | Arg-HCl, | | |
| | | 250 mmol/ | 125 mmol/L | 75 mmol/L | | |
| | | L Sucrose, | Sucrose, 0.25 | Sucrose, | | |
| | | 0.5 mg/mL | mg/mL | 0.15 mg/mL | | |
| | | Poloxamer 188 | Poloxamer 188 | Poloxamer 188 | | |
| HMWS | Initial | 0.52 | 0.49 | 0.52 | 0.47 | 0.44 |
| [%] | 50° C. | 1.31 | 1.29 | 1.30 | 0.62 | 0.47 |
| | after 8 | | | | | |
| | weeks | | | | | |
| Basic | Initial | 14.8 | 14.2 | 13.9 | 14.2 | 14.3 |
| region | 50° C. | 16.5 | 16.6 | 17.0 | 17.0 | 17.3 |
| [%] | after 8 | | | | | |
| | weeks | | | | | |

As is apparent from Table 7, samples comprising tested concentrations of each of the components, i.e., samples comprising 6 mg/mL to 100 mg/mL CIM331, 6 mmol/L to 20 mmol/L Tris-HCl, 45 mmol/L to 150 mmol/L Arg-HCl, 75 mmol/L to 250 mmol/L Sucrose, and 0.15 mg/mL to 0.5 mg/mL Poloxamer 188 had sufficient stability eight weeks after 50° C./75% RH thermal acceleration. Regarding HMWS, when formulation components other than antibody were the same concentrations, samples comprising lower-concentrated antibody showed more excellent stability.

### Example 6

### Effects of Arginine in Suppressing Aggregates and Components With Charge Heterogeneity of Humanized IgG2 Antibody CIM331 During Thermal Accelerated Storage

### [6-1] Stability Assessment of Lyophilized Formulations

### (1) Materials

The antibody described in Example 1 was used.

### (2) Test Samples

Various prepared solutions containing 30 mg/mL CIM331, 6 mmol/L Tris-HCl, pH7, 75 mmol/L sucrose, 0.15 mg/mL Poloxamer 188, and any of 45 mmol/L Arg-HCl, 45 mmol/L histidine, 45 mmol/L lysine-HCl, and 45 mmol/L glycine as a stabilizer, were prepared and filled into glass vials (2 mL/vial). Filled drug solutions were lyophilized under the conditions shown below.

| Temperature | Time | | Pressure |
|---|---|---|---|
| Room temperature → −45° C. | 1 | hour | — |
| −45° C. | 6 | hours | — |
| −45° C. → 0° C. | 50 | minutes | 10 Pa |
| 0° C. | 50 | hours | 10 Pa |
| 0° C. → 5° C. | 30 | minutes | 6 Pa |
| 5° C. | 10 | hours | 6 Pa |

After humanized antibody-containing lyophilized formulations prepared as such were left to stand in a 50° C./75% RH incubator for eight weeks, reconstituted solutions prepared such that the CIM331 concentration became 100 mg/mL by adding ultra-pure water were used as test samples.

### (3) Method for Measuring Amounts of CIM331 Aggregates and Method for Calculating Amounts of CIM331 Aggregates

The methods were performed according to the methods described in Example 1.

### (4) Methods for Measuring and Calculating Components With Charge Heterogeneity of CIM331

The methods were performed according to the methods described in Example 4.

### (5) Results

The obtained results are shown in Table 8.

TABLE 8

| | | Arg- | | | |
|---|---|---|---|---|---|
| Stabilizer | | HCl | Histidine | Lysine-HCl | Glycine |
| HMWS | Initial | 0.49 | 0.45 | 0.51 | 0.56 |
| [%] | 50° C. after 8 | 1.29 | 1.82 | 1.65 | 2.45 |
| | weeks | | | | |
| HMWS increase [%] | | 0.80 | 1.37 | 1.14 | 1.89 |
| Main | Initial | 78.8 | 78.3 | 78.4 | 78.4 |
| region [%] | 50° C. after 8 | 74.8 | 71.4 | 71.2 | 68.2 |
| | weeks | | | | |

| | | | | |
|---|---|---|---|---|
| Main region decrease [%] | 4.0 | 6.9 | 7.2 | 10.2 |

As is apparent from Table 8, a sample added with arginine-hydrochloride showed the highest aggregate-suppressing effect and effect of suppressing components with charge heterogeneity as compared to samples added with histidine, lysine-hydrochloride, or glycine eight weeks after 50° C./75% RH thermal acceleration.

### [6-2] Stability Assessment of Solution Formulations

### (1) Materials

The antibody described in Example 1 was used.

### (2) Test Samples

Various prepared solutions comprising 100 mg/mL CIM331, 20 mmol/L Tris-HCl, pH7, 250 mmol/L Sucrose, 0.50 mg/mL Poloxamer 188, and any of 150 mmol/L Arg-HCl, 150 mmol/L histidine, 150 mmol/L lysine-HCl, and 150 mmol/L glycine as a stabilizer were prepared and filled into glass vials (1 mL/vial). After humanized antibody-containing solution formulations prepared as such were left to stand in a 40° C./75% RH incubator for four weeks, they were used as test samples.

### (3) Methods for Measuring and Calculating Components With Charge Heterogeneity of CIM331

The methods were performed according to the methods described in Example 4.

### (4) Results

The obtained results are shown in Table 9.

TABLE 9

| | | | | Lysine- | |
|---|---|---|---|---|---|
| Stabilizer | | Arg-HCl | Histidine | HCl | Glycine |
| Main | Initial | 78.6 | 78.2 | 78.5 | 78.5 |
| region | 40° C. after 4 | 52.0 | 43.0 | 46.7 | 43.2 |
| [%] | weeks | | | | |

| | | | | |
|---|---|---|---|---|
| Main region decrease [%] | 26.6 | 35.2 | 31.8 | 35.3 |

As is apparent from Table 9, a sample added with arginine-hydrochloride showed the highest effect of suppressing components with charge heterogeneity as compared to samples added with histidine, lysine-hydrochloride, or glycine four weeks after 40° C./75% RH thermal acceleration.

### INDUSTRIAL APPLICABILITY

Formulations of the present invention are formulations with excellent stability both in lyophilized state and in solution state, and characterized in that aggregate formation of proteins such as antibody molecules is suppressed after storage in solution states and after storage in lyophilized states and reconstitution by water. Formulations of the present invention in which deteriorative reaction is difficult to occur as such can be used for. for example. the treatment of atopic dermatitis and such by subcutaneous administration.

1. A solution formulation comprising an IL-31 antagonist as an active ingredient, wherein the solution formulation comprises arginine and/or a salt thereof.

2. A lyophilized formulation comprising an IL-31 antagonist as an active ingredient, wherein the formulation comprises arginine and/or a salt thereof, and sucrose and/or trehalose.

3. The formulation of claim 1 or 2, wherein the arginine and/or salt thereof is arginine-hydrochloride, arginine-aspartate, or arginine-glutamate.

4. The formulation of any one of claims 1 to 3, wherein the molar ratio of the arginine and/or salt thereof to the IL-31 antagonist is 220:1 to 1100:1, and/or the weight ratio of the arginine to the IL-31 antagonist is 0.3:1 to 1.3:1.

5. The lyophilized formulation of any one of claims 2 to 4, wherein, between the sucrose or trehalose and the IL-31 antagonist, the molar ratio is 370:1 to 1840:1 and/or the weight ratio is 0.8:1 to 4.3:1.

6. The formulation of any one of claims 1 to 5, further comprising Tris buffer as a buffer agent.

7. The formulation of any one of claims 1 to 6, further comprising Poloxamer 188 or polysorbate as a nonionic surfactant.

8. The formulation of any one of claims 1 to 7, wherein pH in a solution state is 6 to 8.

9. The formulation of any one of claims 2 to 8, wherein the formulation is a lyophilized formulation which is a composition resulting from lyophilizing a solution comprising:
1 to 200 mg/mL IL-31 antagonist;
1 to 200 mmol/L Tris buffer;
4.5 to 1500 mmol/L arginine or a salt thereof;
7.5 to 2500 mmol/L sucrose or trehalose; and
0.01 to 5 mg/mL Poloxamer 188 or Polysorbate 20,
wherein pH is 6 to 8 when the formulation is reconstituted in water.

10. The formulation of any one of claims 1 to 9, wherein the IL-31 antagonist is an antibody that inhibits IL-31 signaling.

11. The formulation of any one of claims 1 to 10, wherein the IL-31 antagonist is
(1) an anti-IL-31RA antibody comprising an H chain variable region which comprises CDR1 of SEQ ID NO: 1, CDR2 of SEQ ID NO: 2, and CDR3 of SEQ ID NO: 3, and an L chain variable region which comprises CDR1 of SEQ ID NO: 4, CDR2 of SEQ ID NO: 5, and CDR3 of SEQ ID NO: 6;
(2) an anti-IL-31RA antibody comprising the H chain variable region of SEQ ID NO: 7 and the L chain variable region of SEQ ID NO: 8; or
(3) an anti-IL-31RA antibody comprising the H chain of SEQ ID NO: 9 and the L chain of SEQ ID NO: 10.

12. The formulation of any one of claims 2 to 10, wherein the formulation is a lyophilized formulation which is a composition resulting from lyophilizing a solution comprising:
1 to 200 mg/mL IL-31 antagonist;
1 to 200 mmol/L Tris buffer;
4.5 to 1500 mmol/L arginine or a salt thereof;
7.5 to 2500 mmol/L sucrose or trehalose; and
0.01 to 5 mg/mL Poloxamer 188 or Polysorbate 20,
wherein, when the formulation is reconstituted in water,
1 to 200 mg/mL IL-31 antagonist;
1 to 200 mmol/L Tris buffer;
4.5 to 1500 mmol/L arginine or a salt thereof;
7.5 to 2500 mmol/L sucrose or trehalose; and
0.01 to 5 mg/mL Poloxamer 188 or Polysorbate 20
are comprised, and pH is 6 to 8.

13. A method for stabilizing an antibody in an antibody-containing formulation, comprising preparing an antibody-containing solution which comprises arginine and/or a salt thereof, and/or sucrose and/or trehalose, wherein in the solution the molar ratio of the arginine and/or salt thereof to the antibody is 220:1 to 1100:1 and the molar ratio of the sucrose or trehalose to the antibody is 370:1 to 1840:1.

14. A method for suppressing antibody aggregation (aggregate formation) in an antibody-containing formulation, comprising preparing an antibody-containing solution which comprises arginine and/or a salt thereof, and/or sucrose and/or trehalose, wherein in the solution the molar ratio of the arginine and/or salt thereof to the antibody is 220:1 to 1100:1 and the molar ratio of the sucrose or trehalose to the antibody is 370:1 to 1840:1.

15. A method for reducing components with charge heterogeneity in an antibody-containing formulation, comprising preparing an antibody-containing solution which comprises arginine and/or a salt thereof, and/or sucrose and/or trehalose, wherein in the solution the molar ratio of the arginine and/or salt thereof to the antibody is 220:1 to 1100:1 and the molar ratio of the sucrose or trehalose to the antibody is 370:1 to 1840:1.

16. A formulation for injection comprising:
(i) a container;
(ii) a lyophilized formulation contained in the container which comprises:
1 to 800 mg IL-31 antagonist;
0.1 to 40 mg tris(hydroxymethyl)aminomethane;
0.8 to 400 mg arginine;
3 to 1100 mg sucrose or trehalose; and
0.01 to 7 mg Poloxamer 188 or Polysorbate 20,
wherein pH is 6 to 8 when the lyophilized formulation is reconstituted in water; and
(iii) optionally, water for injection for reconstituting the lyophilized formulation.

17. A formulation for injection comprising:
(i) a container,
(ii) a lyophilized formulation contained in the container which comprises:
10 to 80 mg anti-IL-31RA antibody, the antibody comprising an H chain variable region which comprises CDR1 of SEQ ID NO: 1, CDR2 of SEQ ID NO: 2, and CDR3 of SEQ ID NO: 3, and an L chain variable region which comprises CDR1 of SEQ ID NO: 4, CDR2 of SEQ ID NO: 5, and CDR3 of SEQ ID NO: 6;
0.8 to 4 mg tris(hydroxymethyl)aminomethane;
8 to 40 mg arginine-hydrochloride;
30 to 110 mg sucrose; and
0.1 to 0.7 mg Poloxamer 188,
wherein pH is 6 to 8 when the lyophilized formulation is reconstituted in water; and
(iii) optionally, water for injection for reconstituting the lyophilized formulation.


SEQUENCE LISTING (12 sequences)

SEQ ID NO: 1 - protein, 5 aa, synthetic construct
  REGION 1..5: note = An artificially synthesized peptide sequence
GYIMN

SEQ ID NO: 2 - protein, 17 aa, synthetic construct
  REGION 1..17: note = An artificially synthesized peptide sequence
LINPYNGGTDYNPQFQD

SEQ ID NO: 3 - protein, 12 aa, synthetic construct
  REGION 1..12: note = An artificially synthesized peptide sequence
DGYDDGPYTLET

SEQ ID NO: 4 - protein, 11 aa, synthetic construct
  REGION 1..11: note = An artificially synthesized peptide sequence
QASEDIYSFVA

SEQ ID NO: 5 - protein, 7 aa, synthetic construct
  REGION 1..7: note = An artificially synthesized peptide sequence
NAQTEAQ

SEQ ID NO: 6 - protein, 9 aa, synthetic construct
  REGION 1..9: note = An artificially synthesized peptide sequence
QHHYDSPLT

SEQ ID NO: 7 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTDYNPQFQDRVTITADKSTSTAYMELSSLRSEDTAVYYCARDGYDDGPYTLETWGQGTLVTVSS

SEQ ID NO: 8 - protein, 107 aa, synthetic construct
  REGION 1..107: note = An artificially synthesized peptide sequence
DIQMTQSPSSLSASVGDRVTITCQASEDIYSFVAWYQQKPGKAPKLLIYNAQTEAQGVPSRFSGSGSGTDFTLTISSLQPEDFATYYCQHHYDSPLTFGGGTKVEIK

SEQ ID NO: 9 - protein, 445 aa, synthetic construct
  REGION 1..445: note = An artificially synthesized peptide sequence
QVQLVQSGAEVKKPGASVKVSCKASGYTFTGYIMNWVRQAPGQGLEWMGLINPYNGGTDYNPQFQDRVTITADKSTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 10 - protein, 214 aa, synthetic construct
  REGION 1..214: note = An artificially synthesized peptide sequence
DIQMTQSPSSLSASVGDRVTITCQASEDIYSFVAWYQQKPGKAPKLLIYNAQTEAQGVPSRFSGSGSGTDFTLTISSLQPEDFATYYCQHHYDSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 764 aa, Homo sapiens
MCIRQLKFFTTACVCECPQNILSPQPSCVNLGMMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFEIILITSLIGGGLLILIILTVAYGLKKPNKLTHLCWPTVPNPAESSIATWHGDDFKDKLNLKESDDSVNTEDRILKPCSTPSDKLVIDKLVVNFGNVLQEIFTDEARTGQENNLGGEKNGYVTCPFRPDCPLGKSFEELPVSPEIPPRKSQYLRSRMPEGTRPEAKEQLLFSGQSLVPDHLCEEGAPNPYLKNSVTAREFLVSEKLPEHTKGEV

SEQ ID NO: 12 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK